(19) United States
(12) Patent Application Publication
(10) Pub. No.: US 2015/0018227 A1
(43) Pub. Date: Jan. 15, 2015

(54) MICRORNA BIOMARKERS

(71) Applicants: Douglas D. Taylor, Louisville, KY (US); Cicek Gercel-Taylor, Louisville, KY (US)

(72) Inventors: Douglas D. Taylor, Louisville, KY (US); Cicek Gercel-Taylor, Louisville, KY (US)

(73) Assignee: UNIVERSITY OF LOUISVILLE RESEARCH FOUNDATION, INC., Louisville, KY (US)

(21) Appl. No.: 14/237,845

(22) PCT Filed: Aug. 3, 2012
(86) PCT No.: PCT/US2012/049615

§ 371 Date: Sep. 28, 2014

Related U.S. Application Data
(60) Provisional application No. 61/515,620, filed on Aug. 5, 2011.

(51) Int. Cl. C12Q 1/68

(52) U.S. Cl. CPC C12Q 1/6886; C12Q 2600/178; C12Q 2600/106; C12Q 2600/112
USPC 506/9

(57) ABSTRACT

The presently disclosed subject matter provides methods for characterization of and evaluation of treatment and/or progression of a lung cancer or a head and neck cancer by measuring amounts of one or more RNAs present in microvesicles isolated from a biological sample from the subject.

This application claims priority from U.S. Provisional Application Ser. No. 61/515,620 filed Aug. 5, 2011, the entire disclosure of which is incorporated herein by this reference.

### TECHNICAL FIELD

The presently disclosed subject matter relates to methods for characterization of and evaluation of treatment and/or progression of a lung cancer or a head and neck cancer. In particular, the presently-disclosed subject matter relates to methods based on determining amounts of one or more exosome-derived micro-RNAs correlated with a lung cancer or a head and neck cancer in a biological sample from a subject.

### INTRODUCTION

The identification of cancer biomarkers suitable for the early detection and diagnosis of cancer holds great promise to improve the clinical outcome of subjects. It is especially important for subjects presenting vague or no symptoms or with tumors that are relatively inaccessible to physical examination. Despite considerable effort directed at early detection, few reliable and cost-effective screening tests have been developed that can diagnose cancer at an early stage.

Head and neck cancer (HNC) refers to a group of biologically similar cancers originating from the upper aerodigestive tract, including the lip, oral cavity (mouth), nasal cavity, paranasal sinuses, pharynx, and larynx. Head and neck cancers are typically squamous cell carcinomas (SCC), originating from the mucosal lining (epithelium) of these regions. Head and neck cancers often spread to the lymph nodes of the neck, and this is often the first (and sometimes only) manifestation of the disease at the time of diagnosis. Head and neck cancer is strongly associated with certain environmental and lifestyle risk factors, including tobacco smoking, alcohol consumption, UV light and occupational exposures, and certain strains of viruses, such as the sexually transmitted human papillomavirus. These cancers are frequently aggressive in their biologic behavior; patients with these types of cancer often develop a second primary tumor. Head and neck cancer is highly curable if detected early, usually with some form of surgery although chemotherapy and radiation therapy may also play an important role. Although early-stage head and neck cancers (especially laryngeal and oral cavity) have high cure rates, up to 50% of head and neck cancer patients present advanced disease and decreasing prognosis. There are about 40,000 new cases of HNC in the US each year, and over a half million cases worldwide.

Non-small-cell lung carcinoma (NSCLC) comprises any epithelial cancer of the lung other than small-cell lung carcinoma. Lung cancers are mainly observed in tobacco smokers. As they are relatively unresponsive to chemotherapy, NSCLCs are primarily treated by surgical resection. However, neoadjuvant and adjuvant chemotherapy is becoming more common, e.g., cisplatin. Radiation therapy is also used. Chemotherapy is used more often for metastatic disease, e.g., EGFR tyrosine kinase inhibitors such as gefitinib. The most common types of NSCLC are squamous cell carcinoma, large cell carcinoma, and adenocarcinoma. Adenocarcinomas are the most common type of lung cancer in non-smokers.

A need persists for the development of improved biomarkers in nearly all cancers, including head and neck cancer and lung cancer. Blood-based assays remain an attractive goal due to the availability and ease of sample collection. Earlier definitive diagnosis of cancer would facilitate earlier and potentially more effective treatment of patients. For HNC, this is highlighted by the fact that such cancers are highly treatable if discovered early, but may not be detected until the cancer has spread to the nodes. As such, there is an unmet need for new biomarkers that individually, or in combination with other biomarkers or diagnostic modalities, deliver the required sensitivity and specificity for early detection and prognosis of cancer and other proliferative diseases. In particular, simple tests for cancer biomarkers that can be performed on readily-accessible biological fluids are needed.

### SUMMARY

In an aspect, the present invention provides a method for characterizing a cancer in a subject, comprising: a) isolating microvesicles from a biological sample of the subject; b) determining an amount of one or more microRNAs from the isolated microvesicles; and c) comparing the amount of the one or more microRNAs to a reference, wherein the cancer is characterized based on a measurable difference in the amount of the one or more microRNAs from the isolated microvesicles as compared to the reference.

In another aspect, the present invention provides a method for evaluating treatment efficacy and/or progression of a cancer in a subject, comprising: a) isolating microvesicles from a biological sample of the subject; b) determining an amount of one or more microRNAs in the isolated microvesicles; and c) comparing the amount of the one or more microRNAs to a reference, wherein the treatment efficacy and/or progression of the cancer is evaluated based on a measurable difference in the amounts of the one or more microRNAs as compared to the reference.

In another aspect, the present invention provides a method for assessing the presence of one or more microRNAs, comprising: a) isolating microvesicles from a biological sample of the subject; b) determining an amount of one or more microRNAs in the isolated microvesicles; and c) comparing the amount of the one or more microRNAs to a reference, wherein the treatment efficacy and/or progression of the cancer is evaluated based on a measurable difference in the amounts of the one or more microRNAs as compared to the reference.

In another aspect, the present invention provides a method for assessing a presence or an amount of one or more microRNAs of a lung cancer miRNA signature or a head and neck cancer miRNA signature, comprising isolating microvesicles from a biological sample, and determining the presence or the amount of the one or more microRNAs in said microvesicles. In some embodiments the microvesicles are shed from lung cancer or head and neck cancer cells.

The cancer can be a head and neck cancer. In some embodiments, the cancer is a head and neck squamous cell carcinoma. The cancer can be a lung cancer. In some embodiments, the cancer is a lung squamous cell carcinoma. In other embodiments, the cancer is a lung adenocarcinoma. The subject can be a human.

In some embodiments, the reference comprises a level of the one or more microRNAs in one or more samples from one or more individuals without the cancer. When the biological sample comprises tissue, the reference can be a level of the one or more microRNAs in normal adjacent tissue from the subject. In other embodiments, the reference comprises a level of the one or more microRNAs in a sample from the subject taken over a time course. This allows the levels of the one or more microRNAs in the subject to be tracked over time. For example, the reference can be a sample from the subject collected prior to initiation of treatment for the cancer and/or onset of the cancer, and the biological sample can be collected after initiation of the treatment or onset of the cancer. By way of example, an increase over time in microRNAs associated with the cancer may indicate an ineffective treatment or a progression of the cancer, whereas a decrease over time in microRNAs associated with the cancer may indicate an effective treatment or a lack of progression of the cancer.

The biological sample can comprise bodily fluid. For example, the biological sample can comprise milk, blood, serum, plasma, ascites, cyst fluid, pleural fluid, peritoneal fluid, cerebral spinal fluid, tears, urine, saliva, sputum, or combinations thereof.

Various techniques can be used to isolate the microvesicles. Isolation as used herein can refer to partial or complete isolation of the entity (e.g., microvesicles or microRNAs) of interest from other biological materials. In an embodiment, isolating the microvesicles comprises using chromatography, such as size exclusion chromatography. In such cases, isolating the microvesicles may further comprise centrifuging a chromatography fraction comprising the microvesicles. The chromatography fraction can be a void volume fraction. In another embodiment, the microvesicles are isolated by affinity selection using a binding agent to a microvesicle surface antigen. For example, in some embodiments the microvesicles are isolated by immunosorbent capture using an antibody to a cell surface antigen. Examples of the antibody include an anti-epithelial cell adhesion molecule (anti-EpCAM) antibody, an anti-CD9 antibody or an anti-CD63 antibody. In embodiments, chromatography and immunosorbent capture are used in combination. For example, a chromatography fraction comprising microvesicles can be subjected to immunosorbent capture. In some embodiments isolating the microvesicles comprises PEG-precipitation of the microvesicles.

The one or more microRNAs are obtained from the biological sample, e.g., from within the isolated microvesicles. Determining the amount of the one or more microRNAs may comprise labeling the one or more microRNAs. In an embodiment, determining the amount of the one or more microRNAs comprises capturing the one or more microRNAs with one or more polynucleotide probes that each selectively bind the one or more microRNAs. The probes may comprise an array. In another embodiment, determining the amount of the one or more microRNAs comprises using a real-time polymerase chain reaction to quantitate the amount of the one or more microRNAs.

The one or more microRNAs may comprise one or more of let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32 and miR-181c. For example, the one or more microRNAs may comprise one or more of miR-16, miR-181c, miR-25, miR-15b, miR-150, miR-148b, miR-92a, miR-222, miR-96, miR-125a-5p, miR-335 and miR-122. As another example, the one or more microRNAs may comprise one or more of let7a, miR133b, miR122, miR20b, miR335, miR196a, miR125a-5p, miR142-5p, miR96, miR222, miR148b, miR92a, miR214, miR130a, miR29a, miR212, miR124, miR21, miR200c, miR100, miR155, miR181b and miR210. In some embodiments, overexpression of the one or more microRNAs as compared to the reference indicates the presence of cancer in the subject, e.g., a head and neck cancer and/or a lung cancer. The methods can also include determining levels of microRNAs that are underexpressed in the presence of cancer, e.g., a head and neck cancer and/or a lung cancer. The cancer can be a squamous cell carcinoma.

In some embodiments, the methods of the invention further comprise selecting a treatment or modifying a treatment for the cancer based on the amount of the one or more microRNAs determined. The methods of the present invention can be performed in vitro.

In a related aspect, the present invention provides use of a reagent to carry out the methods disclosed herein. The prevent invention further provides a kit comprising a reagent to carry out the methods disclosed herein. In an embodiment, the reagent comprises one or more primer pair for amplifying one or more microRNAs described above.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 is a schematic diagram showing exemplary methodology for chromatographically isolating cancer-derived microvesicles and miRNA from the microvesicles, determining amounts of the miRNA by microarray, and analyzing the data to determine if cancer is present in the subject tested.

FIG. 2 is a schematic diagram showing exemplary methodology for isolating cancer-derived microvesicles and miRNA from the microvesicles, determining amounts of the miRNA by real-time PCR, and analyzing the data to determine if cancer is present and the stage of cancer in the subject tested.

FIG. 3 is a schematic diagram showing exemplary methodology for isolating cancer-derived microvesicles and miRNA from the microvesicles, determining amounts of the miRNA by microarray, and analyzing the data to determine if cancer is present in the subject tested. In the diagram, microvesicles are captured with anti-EpCAM antibodies. Antibodies to other vesicle biomarkers of interest can also be used.

FIG. 4 illustrate absolute CT values from qRT-PCR for specific exosomal miRNAs from patients with Head and Neck Squamous Cell Carcinoma (“HN SCC”) with Lung Squamous Cell Carcinoma (“Lung SCC”) and Lung adenocarcinoma (“Lung Adeno”), as compared to absolute CT values from qRT-PCR for specific microRNAs utilizing small RNA isolated from exosomes isolated from a pool of normal controls (“Control”).

FIGS. 5A-B illustrate a change in exosomal miRNA profiles from representative patients with HNSCC between initial sample and follow-up sample (post treatment) for patients responding (FIG. 5A) versus non-responders (FIG. 5B). From left to right, the microRNAs are let7a, miR133b, miR122, miR20b, miR335, miR196a, miR125a-5p, miR142-5p, miR96, miR222, miR148b, miR92a, miR214, miR130a, miR29a, miR212, miR124, miR21, miR200c, miR100, miR155, miR181b, miR210.

FIGS. 6A-K illustrate a change in exosomal miRNA profiles from representative patients with head and neck cancer between initial sample (“Group 1,” obtained on the day of, but prior to surgery) and follow-up sample (“Group 2,” obtained 3 months post surgery). Following surgery, the patients received either chemotherapy or a combination of chemotherapy and radiation. Control data are also included, providing exosomal miRNA profiles obtained using small RNA isolated from exosomes isolated from a pool of normal controls (“Control”). Results for “responders” are set forth in FIGS. 6A-D, and results for “non-responders” are set forth in FIGS. 6E-K. Patients considered responders have no evidence of disease at 18 months after initial surgery, while non-responders were diagnosed with recurrent disease within 18 months of initial surgery. From left to right, the microRNAs are let7a, miR133b, miR122, miR20b, miR335, miR196a, miR125a-5p, miR142-5p, miR96, miR222, miR148b, miR92a, miR214, miR130a, miR29b, miR212, miR124, miR21, miR200c, miR100, miR155, miR181b, miR210.

### DESCRIPTION OF EXEMPLARY EMBODIMENTS

Over the last 5 years, expression profiling technologies have identified new biomarkers with diagnostic applications. One such biomarker group is a class of small non-coding RNAs, termed microRNAs (miRNAs) (Iorio et al. 2007; De Cecco et al., 2004; Calin & Croce, 2006). MicroRNAs, small (e.g., 17-25 nucleotides in length) non-coding RNAs, suppress the translation of target mRNAs by binding to their 3′ untranslated region (Esquela-Kerscher & Slack, 2006; Bartel, 2004). Post-transcriptional silencing of target genes by miRNA can occur either by cleavage of homologous mRNA or by specific inhibition of protein synthesis.

All tumors analyzed by miRNA profiling have exhibited significantly distinct miRNA signatures, compared with normal cells from the same tissue (Iorio et al. 2007; Calin & Croce, 2006a; Calin & Croce, 2006b). Lu et al. (2005) performed an analysis of leukemias and solid cancers and determined that miRNA-expression profiles could classify human cancers by developmental lineage and differentiation state. The expressions of individual miRNAs and specific miRNA signatures have now been linked to the diagnosis and prognosis of many human cancers.

Using tissue specimens, Iorio et al. (2007) demonstrated that, in comparison to normal ovary, specific miRNAs were aberrantly expressed in ovarian cancer, with miR-141, miR-200a, miR-200b, and miR-200c being the most significantly overexpressed. They further demonstrated the hypomethylation in ovarian tumors resulted in the up-modulation of miR-21, miR-203, and miR-205, compared with normal ovary. Two of these up-modulated miRNAs, miR-200a and miR-200c, were enhanced in all the three histologic types examined (serous, endometrioid, and clear cell), whereas miR-200b and miR-141 up-modulation was shared by endometrioid and serous histologic types. In general, the miRNA signatures obtained comparing different histologic types of ovarian cancers (serous, endometrioid, clear cell, and mixed) with the normal tissue were overlapping in most cases. Their analysis of ovarian tumors also demonstrated the absence of differentially expressed miRNAs in relation to tumor stage or grade, which could have resulted from their set of samples being primarily derived from advanced stage tumors.

Among the miRNAs most significantly up-modulated, miR-200a and miR-141 belong to the same family, miR-200b is localized on chromosome 1p36.33 in the same region as miR-200a and miR-200c is localized on chromosome 12p13.31 in the same region of miR-141 (Iorio et al. (2007)). This association would agree with the findings of Zhang et al. (2006) that proposed that the up-modulation of specific miRNAs could be the amplification of the miRNA genes. Using high-resolution array-based comparative genomic hybridization, an aberrantly high proportion of loci containing miRNA genes exhibited DNA copy number alterations. In ovarian cancer, 37.1% of the genomic loci containing miRNA genes were associated with DNA copy number alterations (Zhang et al., 2006). In breast cancer and melanoma, an even greater proportion of these loci exhibit altered DNA copy numbers (72.8% and 85.9%, respectively) (Zhang et al., 2006). As a result, miRNA expression patterns, or signatures, appear to be more characteristic of the developmental origins of tumors than mRNA expression patterns and may be associated with diagnosis, staging, progression, prognosis, and response to treatment. However, as cancer diagnostic tools, prior to the presently-disclosed subject matter, the analyses of miRNA signatures have been limited to tissue biopsies.

A recently described characteristic of cancer cells is their ability to release or shed intact, vesicular portions of the plasma membrane, known in the art as membrane fragments, membrane vesicles, or microvesicles. Disclosed herein are miRNAs associated with microvesicles originating from cancer cells (i.e., “cancer-derived microvesicles”). The presently disclosed subject matter further discloses that miRNA isolated from cancer-derived microvesicles exhibits expression levels in subjects suffering from cancer that differ (e.g., increased or decreased) from miRNA expression levels measured in subjects free of cancer (referred to herein as “miRNA control levels”). Further, the presently disclosed subject matter provides for the isolation of cancer-derived microvesicles from readily-accessible biological fluids from a test subject. As such, the presently disclosed subject matter provides methods for diagnosis and prognosis of cancer based on the collection and measurement of cancer-derived microvesicle miRNA levels from readily-accessible biological samples, and without necessitating direct sampling of cancer cells.

“Exosomes” are microvesicles released from a variety of different cells, including cancer cells (i.e., “cancer-derived exosomes”). These small vesicles (50-100 nm in diameter) derive from large multivesicular endosomes and are secreted into the extracellular milieu. The precise mechanisms of exosome release/shedding remain unclear; however, this release is an energy-requiring phenomenon, modulated by extracellular signals. They appear to form by invagination and budding from the limiting membrane of late endosomes, resulting in vesicles that contain cytosol and that expose the extracellular domain of membrane-bound cellular proteins on their surface. Using electron microscopy, studies have shown fusion profiles of multivesicular endosomes with the plasma membrane, leading to the secretion of the internal vesicles into the extracellular environment. The rate of exosome release is significantly increased in most neoplastic cells and occurs continuously. Increased release of exosomes and their accumulation appear to be important in the malignant transformation process. In addition to cancer cells, the release of exosomes has also been demonstrated to be associated with cells of embryonic origin (such as the placenta) and activated lymphoid cells.

Although extracellular shedding of exosomes occurs in other types of cells, under specific physiological conditions, the accumulation of exosomes from non-neoplastic cells is rarely observed in vivo. In contrast, exosomes released by tumor cells accumulate in biologic fluids, including in sera, ascites, and pleural fluids. Exosome release and its accumulation appear to be important features of the malignant transformation. Shed cancer-derived exosomes do not necessarily mirror the general composition of the plasma membrane of the originating tumor cell, but represent “micromaps,” with enhanced expression of tumor antigens.

The release of exosomes appears to be an important feature of intercellular communication. Since released exosomes express molecules with biologic activity (such as Fas ligand, PD-1, MICA/B, mdr1, MMPs, CD44, and autoreactive antigens), the ability of these microvesicles to modulate lymphocyte and monocyte functions have been analyzed in several models. It has been theorized that these released exosomes modulate lymphocyte functions by mimicking “activation induced cell death” (AICD). Lymphoid cells appear to release exosomes following activation and these appear to play an essential role in immunoregulation, by preventing excessive immune responses and the development of autoimmunity. It has been postulated that exosome release by tumor cells is a re-expression of the fetal cell exosomes and that both constitute pathways to circumvent immunosurveillance.

MicroRNAs are naturally occurring, small non-coding RNAs that are about 17 to about 25 nucleotide bases (nt) in length in their biologically active form. miRNAs post-transcriptionally regulate gene expression by repressing target mRNA translation. It is thought that miRNAs function as negative regulators, i.e. greater amounts of a specific miRNA will correlate with lower levels of target gene expression.

There are three forms of miRNAs existing in vivo, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nt overhang at the 3′ end. The cleavage product, the premature miRNA (pre-miRNA) is about 60 to about 110 nt long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length.

MicroRNAs function by engaging in base pairing (perfect or imperfect) with specific sequences in their target genes' messages (mRNA). The miRNA degrades or represses translation of the mRNA, causing the target genes' expression to be post-transcriptionally down-regulated, repressed, or silenced. In animals, miRNAs do not necessarily have perfect homologies to their target sites, and partial homologies lead to translational repression, whereas in plants, where miRNAs tend to show complete homologies to the target sites, degradation of the message (mRNA) prevails.

MicroRNAs are widely distributed in the genome, dominate gene regulation, and actively participate in many physiological and pathological processes. For example, the regulatory modality of certain miRNAs is found to control cell proliferation, differentiation, and apoptosis; and abnormal miRNA profiles are associated with oncogenesis. Additionally, it is suggested that viral infection causes an increase in miRNAs targeted to silence “pro-cell survival” genes, and a decrease in miRNAs repressing genes associated with apoptosis (programmed cell death), thus tilting the balance towards gaining apoptosis signaling.

Thousands of mRNA are under this selection pressure by hundreds of miRNA species identified so far. This selection process is instrumental in dampening specific groups of gene expressions which, for example, may no longer be needed, to allow cells to channel their physiological program direction to a new pathway of gene expression. The miRNA-dependent dampening of target groups of gene expression is a robust and rapid regulation to allow cells to depart from an old program and transition to a new program. A typical example of this is demonstrated during embryonic development, when a particular group of cells is directed to become unique specialized cell types such as neurons, cardiomyocytes, muscle, etc.

It is thought that expression levels of roughly a third of human genes are regulated by miRNAs, and that the miRNA regulation of unique gene expressions is linked to the particular signaling pathway for each specific cell type. For example, the apoptosis signaling pathway may be dictated by a group of miRNAs targeted to destabilize pro-survival gene messages, allowing alternative pro-apoptosis genes to gain dominance and thus activate the death program. Another example is the control of cancer growth; a recent discovery has shown that miRNAs may also be essential in preventing cells from becoming neoplastic. For example, two oncogenes, cMyc and cRas, are found to share control by one miRNA species, whose expression is down-regulated in cancer. In other words, lack of this miRNA allows the unchecked expression of cMyc and cRas, thus permitting these two genes to become abundantly present in cancer cells, allowing them to acquire uncontrolled cell proliferating ability, and set the stage for neoplastic growth. Additionally, it has been reported that a miRNA mutation is responsible for a phenotype of muscularity in sheep of Belgian origin, suggesting that mutations associated with genetic disorders could be found in miRNAs, where no evidence of mutations have been found in promoter regions, coding areas, and slicing sites.

It is possible that a coordinated orchestration of multiple pathways serves to control a particular cellular state, wherein certain molecular “hubs” may be involved, which are functionally manipulated by hierarchical orders and redundancy of molecular control. Indeed, dozens of miRNAs may operate to ensure that these “hubs” can exert either major or minor functions in cells, by simply repressing the expression of either themselves or their functional opponents. Thus, one gene product may function as a major “hub” for one signaling pathway in one type of cell, and in another cell type, it may be a minor “hub,” or may not be used at all. MicroRNA control of “hub” gene expressions may then be an expedient mechanism to provide such versatility for various molecules to serve as either major or minor “hubs,” or not at all, for different types of cellular operational modalities.

Given the role of miRNAs in gene regulation, and in many physiological and pathological processes, information about their interactive modes and their expression patterns is desirable to obtain. Systems and methods of quantitating and identifying which groups of putative miRNAs are in operation in a particular cell type, or in association with a particular process or condition of interest, can provide information useful for understanding how each cellular state evolves and is maintained, and how dysfunctional maintenance is abetted by improper decreases or increases of unique sets of miRNAs to regulate the expression of key genes. Such understanding can prove useful in the diagnosis and characterization of a number of disorders, including cancer.

As potential clinical diagnostic tools miRNAs have been shown to be important and accurate determinants for many if not all cancers. Increasing evidence shows that expression of miRNA genes is deregulated in human cancer. The expression of miRNAs is highly specific for tissues and developmental stages and has allowed recently for molecular classification of tumors. To date, all tumors analyzed by miRNA profiling have shown significantly different miRNA profiles compared with normal cells from the same tissue. Flow-cytometric miRNA profiling demonstrated that miRNA-expression profiles classify human cancers according to the developmental lineage and differentiation state of the tumors. Specific over- or underexpression has been shown to correlate with particular tumor types. MicroRNA overexpression could result in down-regulation of tumor suppressor genes, whereas their underexpression could lead to oncogene up-regulation. Using large-scale microarray analysis, cancer cells have shown distinct miRNA profiles compared with normal cells with some miRNA genes overexpressed and other miRNAs downregulated in cancer cells versus normal cells. Hierarchical clustering analyses showed that miRNA signatures enable the tumor samples to be grouped on the basis of their tissue of origin. Genome-wide profiling studies have been performed on various cancer types, including CLL, breast cancer, glioblastoma, thyroid papillary carcinoma, hepatocellular carcinoma, ovarian cancer, colon cancer, and endocrine pancreatic tumors. In a study of 104 matched pairs of primary cancerous and non-cancerous ovarian tissue, 43 differentially expressed miRNAs were observed; 28 were downregulated and 15 were overexpressed in tumors.

Statistical analyses of microarray data obtained by two different methods, significance analysis of microarrays (SAM) and prediction analysis of microarrays (PAM) from six solid tumors (ovarian, breast, colon, gastric and prostate carcinomas and endocrine pancreatic tumors), demonstrated a common signature composed of miRNAs differentially expressed in at least three tumor types. At the top of the list were miR-21, which was overexpressed in six types of cancer cells, and miR-17-5p and miR-191, which were overexpressed in five. As the embryological origin of the analyzed tumors was different, the significance of such findings could be that these common miRNAs participate in fundamental signaling pathways altered in many types of tumor. Supporting the function of these genes in tumorigenesis, it was found that the predicted targets for the differentially expressed miRNAs are significantly enriched for those that target known tumor suppressors and oncogenes. Furthermore, miR-21, the only miRNA overexpressed in all six types of cancer analyzed was shown to directly target the tumor suppressor PTEN, which encodes a phosphatase inhibiting growth and/or survival pathways. The function of PTEN is altered in advanced tumors of various types, including breast, ovarian, gastric and prostate.

In some embodiments of the presently disclosed subject matter, a method for characterizing a cancer in a subject is provided. Characterizing can include providing a diagnosis, prognosis, and/or theragnosis of the cancer. In some embodiments of the presently-disclosed subject matter, a method for evaluation treatment efficacy and/or progression of a cancer in a subject is provide. In some embodiments of the presently-disclosed subject matter, a method for assessing the presence of one or more microRNAs of a cancer (e.g., a miR signature or miR expression profile) is provided. In some embodiments, the cancer is a lung cancer. In some embodiments, the cancer is a head and neck cancer.

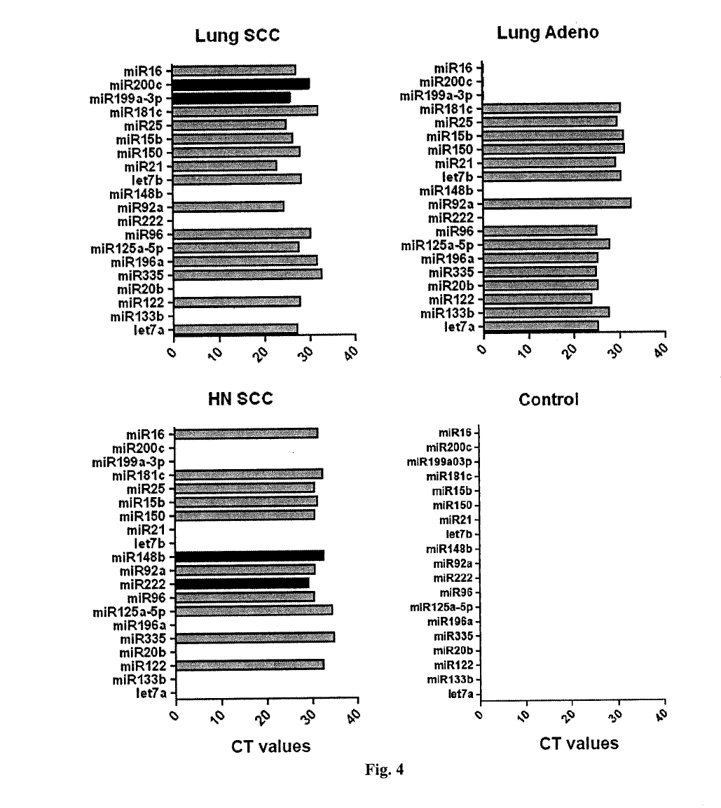

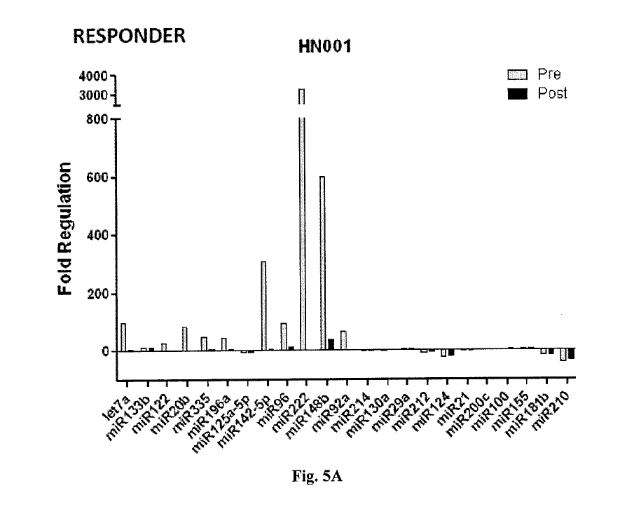

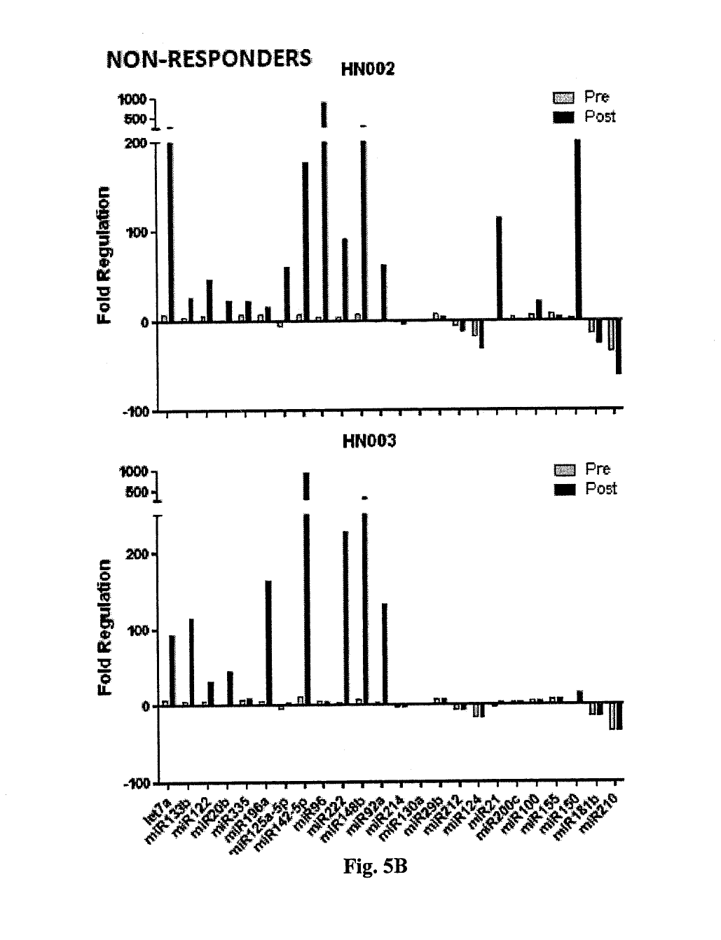

In some embodiments, the method comprises providing a biological sample from a subject; isolating cancer-derived microvesicles comprising miRNAs from the biological sample; determining an amount of one or more of the miRNAs. In some embodiments, the method further includes comparing the amount of the one or more miRNAs to one or more miRNA control levels. The subject can then be diagnosed as having the cancer if there is a measurable difference in the amount of the one or more miRNAs from the cancer-derived microvesicles in the biological sample as compared to the one or more control levels. A non-limiting list of exemplary miRNAs that can be measured are provided in Example 2, e.g., let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32, and miR-181c, and/or as provided in FIGS. 4 and 5. In some embodiments, the miRNAs measured are selected from the miRNAs listed in FIGS. 4 and 5, and in some particular embodiments, the miRNAs measured are miRNAs selected from the group consisting of miR-16, miR-181c, miR-25, miR-15b, miR-150, miR-148b, miR-92a, miR-92b, miR-222, miR-96, miR-125a-5p, miR-335 and miR-122. The miRNAs measured can also be miRNAs selected from the group consisting of let7a, miR133b, miR122, miR20b, miR335, miR196a, miR125a-5p, miR142-5p, miR96, miR222, miR148b, miR92a, miR214, miR130a, miR29a, miR212, miR124, miR21, miR200c, miR100, miR155, miR181b and miR210. These miRs can be measured to detect a cancer such as those described herein, e.g., a head and neck cancer and/or a lung cancer. In some embodiments, the cancer comprises squamous cell carcinoma. As will be recognized by one or ordinary skill in the art, in some embodiments, methods of the presently-disclosed subject matter can be performed in vitro.

The term “cancer” refers to all types of cancer or neoplasm or malignant tumors found in animals, including leukemias, carcinomas, adenomas and sarcomas. Examples of cancers are cancer of the brain, bladder, breast, cervix, colon, head and neck, kidney, lung, non-small cell lung, melanoma, mesothelioma, ovary, pancreas, prostate, sarcoma, stomach, and uterus.

The term “leukemia” includes progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The term “squamous cells” refers to the epithelium (tissue layer) that is the surface cells of much of the body. For example, skin and mucous membranes are squamous cells. Squamous cell neoplasms include without limitation papillary carcinoma, verrucous squamous cell carcinoma, papillary squamous cell carcinoma, squamous cell carcinoma, large cell keratinizing squamous cell carcinoma, small cell keratinizing squamous cell carcinoma, spindle cell squamous cell carcinoma, adenoid/pseudoglandular squamous cell carcinoma, intraepidermal squamous cell carcinoma, lymphoepithelial carcinoma, basaloid squamous cell carcinoma, clear cell squamous cell carcinoma, keratoacanthoma, signet ring cell squamous cell carcinoma, and spindle cell squamous cell carcinoma. Squamous cell carcinoma is one of the most common cancers in humans, and usually arises from mutated ectodermal or endodermal cells lining body cavities. It can develop in a variety of organs and tissues, including the skin, lips, mouth, esophagus, urinary bladder, prostate, lung, vagina, cervix, and others. Squamous cell carcinoma is most likely to appear in males over 40 years of age with a history of heavy alcohol use coupled with smoking. Head and neck squamous cell carcinoma (HNSCC) is the most common form of larynx cancer, accounting for over 90% of throat cancer. Squamous cell lung carcinoma is a type of non-small-cell lung carcinoma (NSCLC) and is closely correlated with a history of tobacco smoking.

In some embodiments, a method for characterizing a lung cancer or a head and neck cancer in a subject is provided and includes isolating microvesicles from a biological sample of the subject; determining a presence or an amount of one or more microRNAs from the isolated microvesicles; and comparing the presence or the amount of the one or more microRNAs to a reference, wherein the lung cancer or the head and neck cancer is characterized based on a measurable difference in the presence or the amount of the one or more microRNAs from the isolated microvesicles as compared to the reference. In some embodiments, the characterizing comprises providing a diagnosis, prognosis and/or theragnosis of the cancer.

In some embodiments, a method for evaluating treatment efficacy and/or progression of a lung cancer or a head and neck cancer in a subject is provided and includes isolating microvesicles from a biological sample of the subject; determining a presence or an amount of one or more microRNAs in the isolated microvesicles; and comparing the presence or the amount of the one or more microRNAs to a reference, wherein the treatment efficacy and/or progression of the lung cancer or the head and neck cancer is evaluated based on a measurable difference in the presence or the amount of the one or more microRNAs as compared to the reference.

In some embodiments, a method for assessing the presence of one or more microRNAs of a lung cancer miRNA signature or a head and neck cancer miRNA signature is provided and includes isolating cancer-derived, extracellular microvesicles from a biological sample; and determining a presence of one or more microRNAs in said microvesicles. In some embodiments the microvesicles are shed from lung cancer or head and neck cancer cells.

In some embodiments, methods of the presently-disclosed subject matter include determining an expression profile or a signature of two or more microRNAs. In some embodiments, the methods can include comparing the expression profile with a profile from a selected reference sample to determine the presence or the amount of two or more microRNAs in said microvesicles.

A biomarker expression profile or biomarker signature for a sample can include information about the identities of biomarkers contained in the sample, quantitative levels of biomarkers contained in the sample, and/or changes in quantitative levels of biomarkers relative to another sample or control. For example, a biomarker signature or profile for a sample can include information about the identities, quantitative levels, and/or changes in quantitative levels of biomarkers from an cancer-derived extracellular microvesicles from a biological sample of particular subject. In some embodiments, a biomarker signature or profile relates to information about two or more biomarkers in a sample (e.g., biomarker signature or profile consisting of 2 biomarkers). In some embodiments, a biomarker signature or profile consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 biomarkers.

In some embodiments, the one or more microRNAs include one or more microRNAs selected from the group consisting of: let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32, and miR-181c. In some embodiments, overexpression of the one or more microRNAs as compared to the reference indicates the presence of cancer in the subject.

In some embodiments, the one or more microRNAs include one or more microRNAs selected from the group consisting of: miR-16, miR-181c, miR-25, miR-15b, miR-150, miR-148b, miR-92a, miR-222, miR-96, miR-125a-5p, miR-335 and miR-122. In some embodiments, the overexpression of the one or more microRNAs as compared to the reference indicates the presence of a head and neck cancer in the subject. In some embodiments, overexpression of the one or more microRNAs as compared to the reference indicates the presence of a head and neck squamous cell carcinoma in the subject.

In some embodiments, the one or more microRNAs include one or more microRNAs selected from the group consisting of: let7a, miR133b, miR122, miR20b, miR335, miR196a, miR125a-5p, miR96, miR92a, let 7b, miR21, miR150, miR15b, miR25, miR181c, miR199a-3p, miR200c, and miR16. In some embodiments, overexpression of the one or more microRNAs as compared to the reference indicates the presence of a lung cancer in the subject.

In some embodiments, the one or more microRNAs include one or more microRNAs selected from the group consisting of: let7a, miR133b, miR122, miR20b, miR335, miR196a, miR125a-5p, miR96, miR92a, let 7b, miR21, miR150, miR15b, miR25, and miR181c. In some embodiments, overexpression of the one or more microRNAs as compared to the reference indicates the presence of a lung adenocarcinoma in the subject.

In some embodiments, the one or more microRNAs include one or more microRNAs selected from the group consisting of: let7a, miR122, miR335, miR196a, miR125a-5p, miR96, miR92a, let 7b, miR21, miR150, miR15b, miR25, miR181c, miR199a-3p, miR200c, and miR16. In some embodiments, overexpression of the one or more microRNAs as compared to the reference indicates the presence of a lung squamous cell carcinoma in the subject.

In some embodiments of the presently-disclosed subject matter include selecting a treatment or modifying a treatment for the cancer based on the amount of the one or more microRNAs determined. As used herein, the terms treatment or treating relate to any treatment of a cancer of interest, including but not limited to prophylactic treatment and therapeutic treatment. As such, the terms treatment or treating include, but are not limited to: preventing a cancer of interest or the development of a cancer of interest; inhibiting the progression of a cancer of interest; arresting or preventing the development of a cancer of interest; reducing the severity of a cancer of interest; ameliorating or relieving symptoms associated with a cancer of interest; and causing a regression of the cancer of interest or one or more of the symptoms associated with the cancer of interest.

In some embodiments of the presently-disclosed subject matter, a method includes comparison to a reference. The reference can include, for example, a level of the one or more microRNAs in one or more samples from one or more individuals without the cancer. In some embodiments, the reference includes a level of the one or more microRNAs in a sample from the subject taken over a time course. In some embodiments, the reference includes a sample from the subject collected prior to initiation of treatment for the cancer and/or onset of the cancer and the biological sample is collected after initiation of the treatment or onset of the cancer.

In some embodiments, the reference can include a standard sample. Such a standard sample can be a reference that provides amounts of one or more microRNAs at levels considered to be control levels. For example, a standard sample can be prepared with to mimic the amounts or levels of one or more microRNAs in one or more samples (e.g., an average of amounts or levels from multiple samples) from one or more individuals without the cancer of interest. In some embodiments the standard sample can be a reference that provides amounts of one or more microRNAs at levels considered to associated with a particular type of cancer and/or a responder or non-responder to treatment.

In some embodiments, the reference can include control data. Control data, when used as a reference, can comprise compilations of data, such as may be contained in a table, chart, graph, e.g., standard curve, or database, which provides amounts or levels of one or more microRNAs considered to be control levels. Such data can be compiled, for example, by obtaining amounts or levels of one or more microRNAs in one or more samples (e.g., an average of amounts or levels from multiple samples) from one or more individuals without the cancer of interest.

The term “biological sample” as used herein refers to a sample that comprises a biomolecule and/or is derived from a subject. Representative biomolecules include, but are not limited to total DNA, RNA, miRNA, mRNA, and polypeptides. The biological sample can be used for the detection of the presence and/or expression level of a miRNA of interest associated with cancer-derived microvesicles. Any cell, group of cells, cell fragment, or cell product can be used with the methods of the presently claimed subject matter, although biological fluids and organs that would be predicted to contain cancer-derived microvesicles exhibiting differential expression of miRNAs as compared to normal controls are best suited. In some embodiments, the biological sample is a relatively easily obtained biological sample, such as for example blood or a component thereof. In some embodiments, the biological sample comprises milk, blood, serum, plasma, ascites, cyst fluid, pleural fluid, peritoneal fluid, cerebral spinal fluid, tears, urine, saliva, sputum, or combinations thereof.

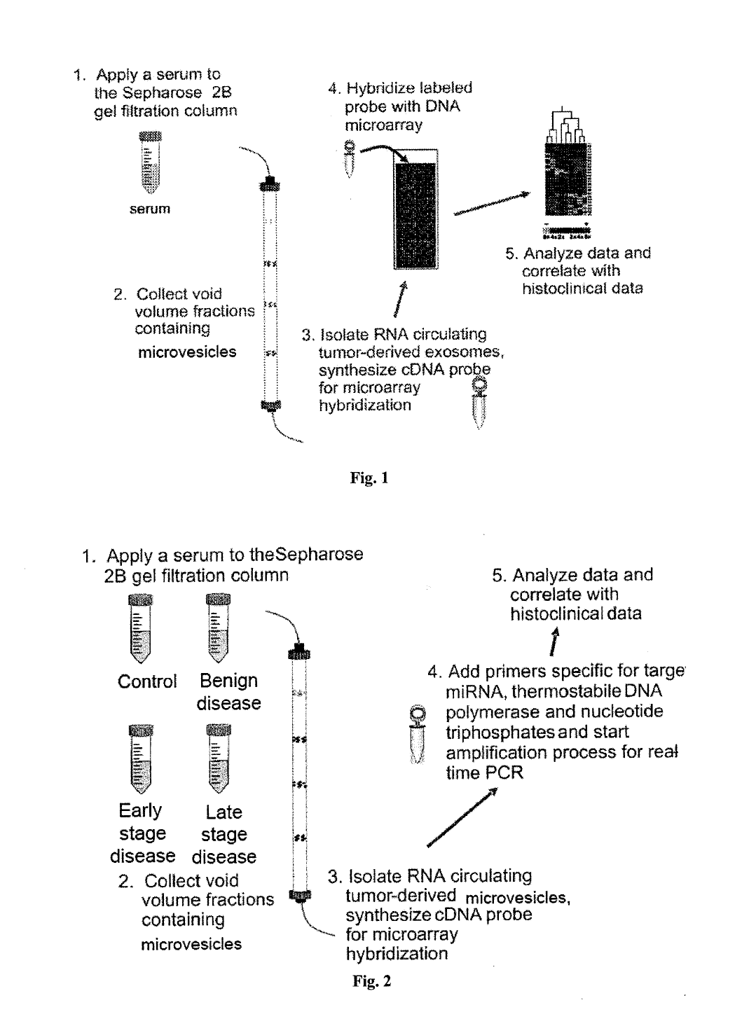

In some embodiments, size exclusion chromatography is used to isolate the cancer-derived microvesicles. See, e.g., FIGS. 1 and 2. Size exclusion chromatography techniques are known in the art. Exemplary, non-limiting techniques are provided in the present Examples. In some embodiments, a void volume fraction is isolated and comprises the microvesicles of interest. Further, in some embodiments, the cancer-derived microvesicles can be further isolated after chromatographic separation by centrifugation techniques (of one or more chromatography fractions), as is generally known in the art. In some embodiments, for example, density gradient centrifugation can be used to further isolate the microvesicles. Still further, in some embodiments, it can be desirable to further separate the cancer-derived isolated microvesicles from microvesicles of other origin.

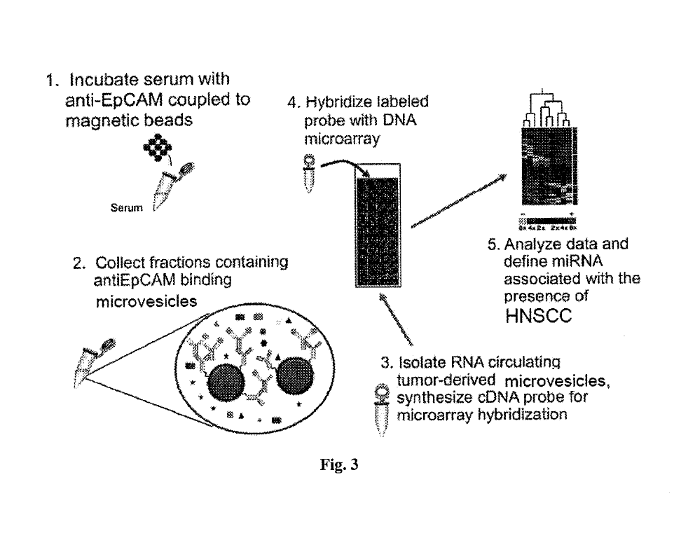

The term “affinity selection”, as used herein refers to the selection of a particular ligand, molecule, substance, or the like based on its affinity for a particular molecule. For example, in some embodiments affinity selection comprises a method for selecting, and thereby isolating, particular microvesicles based on their affinity for particular binding agents. In this regard, the term “binding agent” is used herein to refer to any agent that has known binding affinities. For example, a binding agent can be an antibody or an aptamer. Thus, binding agents can be used in affinity selection to select particular ligands, molecules, substances, or the like based on the extent to which they bind with a particular binding agent. In some embodiments, affinity selection comprises separating the cancer-derived microvesicles from non-cancer-derived microvesicles by immunosorbent capture using an anti-cancer antigen antibody as the binding agent. See, e.g., FIG. 3. Exemplary anti-cancer antigen antibodies include, but are not limited to, anti-epithelial cell adhesion molecule (anti-EpCAM) antibodies, used as, for example, set forth in the present Examples.

The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a biomarker (e.g., an miRNA expression level), the amount (including presence or absence) of which is indicative of the presence, severity, or absence of the condition.

Along with diagnosis, clinical cancer prognosis is also an area of great concern and interest. It is important to know the aggressiveness of the cancer cells and the likelihood of tumor recurrence in order to plan the most effective therapy. Some cancers, for example, are managed by several alternative strategies. In some cases local-regional and systemic radiation therapy is used while in other cases surgical intervention and/or chemotherapy are employed. Current treatment decisions for individual cancer subjects can be based on (1) the number of lymph nodes involved with disease, (2) cancer marker(s) status, (3) the size of the primary tumor, and (4) stage of disease at diagnosis. However, even with these factors, accurate prediction of the course of disease for all cancer subjects is not possible. If a more accurate prognosis can be made, appropriate therapy, and in some instances less severe therapy, for the patient can be chosen. Measurement of cancer-derived microvesicle miRNA levels disclosed herein can be useful in order to categorize subjects according to advancement of cancer who will benefit from particular therapies and differentiate from other subjects where alternative or additional therapies can be more appropriate. Treatment related diagnostics are sometimes referred to as “theranosics.” As such, in some embodiments of the presently disclosed subject matter, a method for characterizing a cancer in a subject is provided. In some embodiments, the method comprises providing a biological sample from a subject; isolating cancer-derived microvesicles comprising micro-RNAs (miRNAs) from the biological sample; determining an amount of one or more of the miRNAs; and comparing the amount of the one or more miRNAs to one or more miRNA control levels. In such embodiments, the cancer can be characterized based on a measurable difference in the amount of the one or more miRNAs from the cancer-derived microvesicles as compared to the one or more miRNA control levels. In some embodiments, characterizing the cancer comprises determining a type, a grade, and/or a stage of the cancer.

“Making a diagnosis” or “diagnosing,” as used herein, are further inclusive of making a prognosis, which can provide for predicting a clinical outcome (with or without medical treatment), selecting an appropriate treatment (or whether treatment would be effective), or monitoring a current treatment and potentially changing the treatment, based on the measure of cancer-derived microvesicle diagnostic miRNA levels. Diagnostic testing that involves treatment, such as treatment monitoring or decision making can be referred to as “theranosis.” Further, in some embodiments of the presently disclosed subject matter, multiple determination of amounts of one or more miRNAs over time can be made to facilitate diagnosis (including prognosis), evaluating treatment efficacy, and/or progression of a cancer. A temporal change in one or more cancer-derived microvesicle miRNA levels (i.e., miRNA amounts in a biological sample) can be used to predict a clinical outcome, monitor the progression of the cancer, and/or efficacy of administered cancer therapies. In such an embodiment for example, one could observe a decrease in the amount of particular miRNAs in a biological sample over time during the course of a therapy, thereby indicating effectiveness of treatment.

The presently disclosed subject matter further provides in some embodiments a method for theranostic testing, such as evaluating treatment efficacy and/or progression of a cancer in a subject. In some embodiments, the method comprises providing a series of biological samples over a time period from the subject; isolating cancer-derived microvesicles comprising miRNAs from the series of biological samples; determining an amount of one or more of the miRNAs in each of the biological samples from the series; and determining any measurable change in the amounts of the one or more miRNAs in each of the biological samples from the series to thereby evaluate treatment efficacy and/or progression of the cancer in the subject. Any changes in the amounts of measured miRNAs over the time period can be used to predict clinical outcome, determine whether to initiate or continue the therapy for the cancer, and whether a current therapy is effectively treating the cancer. For example, a first time point can be selected prior to initiation of a treatment and a second time point can be selected at some time after initiation of the treatment. miRNA levels can be measured in each of the samples taken from different time points and qualitative and/or quantitative differences noted. A change in the amounts of one or more of the measured miRNA levels from the first and second samples can be correlated with prognosis, theranosis, determining treatment efficacy, and/or progression of the disease in the subject.

The terms “correlated” and “correlating,” as used herein in reference to the use of diagnostic and prognostic miRNA levels associated with cancer, refer to comparing the presence or quantity of the miRNA levels in a subject to its presence or quantity in subjects known to suffer from a cancer, or in subjects known to be free of the cancer, i.e. “normal subjects” or “control subjects.” For example, a level of one or more miRNAs in a biological sample can be compared to a miRNA level for each of the specific miRNAs tested and determined to be correlated with a cancer. The sample's one or more miRNA levels is said to have been correlated with a diagnosis; that is, the skilled artisan can use the miRNA level(s) to determine whether the subject suffers from the cancer and respond accordingly. Alternatively, the sample's miRNA level(s) can be compared to control miRNA level(s) known to be associated with a good outcome (e.g., the absence of cancer), such as an average level found in a population of normal subjects.

In certain embodiments, a diagnostic or prognostic miRNA level is correlated to a cancer by merely its presence or absence. In other embodiments, a threshold level of a diagnostic or prognostic miRNA level can be established, and the level of the miRNA in a subject sample can simply be compared to the threshold level.

As noted, in some embodiments, multiple determinations of one or more diagnostic or prognostic miRNA levels can be made, and a temporal change in the levels can be used to determine a diagnosis or prognosis. For example, specific miRNA level(s) can be determined at an initial time, and again at a second time. In such embodiments, an increase in the miRNA level(s) from the initial time to the second time can be diagnostic of the cancer, or a given prognosis. Likewise, a decrease in the miRNA level(s) from the initial time to the second time can be indicative of the cancer, or a given prognosis. Furthermore, the degree of change of one or more miRNA level(s) can be related to the severity of the cancer and/or timeline of disease progression and future adverse events.

The skilled artisan will understand that, while in certain embodiments comparative measurements can be made of the same miRNA level(s) at multiple time points, one can also measure given miRNA level(s) at one time point, and second miRNA level(s) at a second time point, and a comparison of these levels can provide diagnostic information.

The phrase “determining the prognosis” as used herein refers to methods by which the skilled artisan can predict the course or outcome of a condition in a subject. The term “prognosis” can refer to the ability to predict the course or outcome of a condition with up to 100% accuracy, or predict that a given course or outcome is more or less likely to occur based on the presence, absence or levels of a biomarker. The term “prognosis” can also refer to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the miRNA level(s) or expressing miRNA level(s) at a reduced level), the chance of a given outcome (e.g., suffering from cancer) may be very low (e.g., <1%), or even absent. In contrast, in individuals exhibiting the condition (e.g., expressing the miRNA level(s) or expressing miRNA level(s) at a level greatly increased over a control level), the chance of a given outcome (e.g., suffering from a form/stage of cancer) may be higher. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome can be performed using statistical analysis. For example, miRNA level(s) (e.g., quantity of one or more miRNAs in a sample) of greater or less than a control level in some embodiments can signal that a subject is more likely to suffer from a cancer than subjects with a level less than or equal to the control level, as determined by a level of statistical significance. Additionally, a change in miRNA level(s) from baseline levels can be reflective of subject prognosis, and the degree of change in marker level can be related to the severity of adverse events. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001. When performing multiple statistical tests, e.g., determining differential expression of a panel of miRNA levels, p values can be corrected for multiple comparisons using techniques known in the art.

In other embodiments, a threshold degree of change in the level of a prognostic or diagnostic miRNA level(s) can be established, and the degree of change in the level of the indicator in a biological sample can simply be compared to the threshold degree of change in the level. A preferred threshold change in the level for miRNA level(s) of the presently disclosed subject matter is about 5%, about 10%, about 15%, about 20%, about 25%, about 30%, about 50%, about 60%, about 75%, about 100%, or about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

The identity and relative quantity of miRNAs in a sample can be used to provide miRNA profiles for a particular sample. An miRNA profile for a sample can include information about the identities of miRNAs contained in the sample, quantitative levels of miRNAs contained in the sample, and/or changes in quantitative levels of miRNAs relative to another sample. For example, an miRNA profile for a sample can include information about the identities, quantitative levels, and/or changes in quantitative levels of miRNAs associated with a particular cancer.

Further with respect to the diagnostic methods of the presently disclosed subject matter, a preferred subject is a vertebrate subject. A preferred vertebrate is warm-blooded; a preferred warm-blooded vertebrate is a mammal. A mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

As noted hereinabove, the presently disclosed subject matter provides for the determination of the amount of cancer-derived microvesicle miRNAs correlated with cancer within biological fluids of a subject, and in particular, from serological samples from a subject, such as for example blood. This provides the advantage of biological samples for testing that are easily acquired from the subject. The amount of one or more miRNAs of interest in the biologic sample can then be determined using any of a number of methodologies generally known in the art and compared to miRNA control levels.

The “amount” of one or more miRNAs determined refers to a qualitative (e.g., present or not in the measured sample) and/or quantitative (e.g., how much is present) measurement of the one or more miRNAs. The “control level” is an amount (including the qualitative presence or absence) or range of amounts of one or more miRNAs found in a comparable biological sample in subjects not suffering from cancer. As one non-limiting example of calculating the control level, the amount of one or more miRNAs of interest present in a normal biological sample (e.g., blood) can be calculated and extrapolated for whole subjects.

An exemplary methodology for measuring miRNA levels from microvesicles in a biological sample is microarray technique, which is a powerful tool applied in gene expression studies. The technique provides many polynucleotides with known sequence information as probes to find and hybridize with the complementary strands in a sample to thereby capture the complementary strands by selective binding. FIGS. 1 and 3 provide flowcharts of exemplary protocols for isolating and measuring microvesicle-derived miRNAs by microarray.

The term “selective binding” as used herein refers to a measure of the capacity of a probe to hybridize to a target polynucleotide with specificity. Thus, the probe comprises a polynucleotide sequence that is complementary, or essentially complementary, to at least a portion of the target polynucleotide sequence. Nucleic acid sequences which are “complementary” are those which are base-pairing according to the standard Watson-Crick complementarity rules. As used herein, the term “complementary sequences” means nucleic acid sequences which are substantially complementary, as can be assessed by the same nucleotide comparison set forth above, or as defined as being capable of hybridizing to the nucleic acid segment in question under relatively stringent conditions such as those described herein. A particular example of a contemplated complementary nucleic acid segment is an antisense oligonucleotide. With regard to probes disclosed herein having binding affinity to miRNAs, the probe can be 100% complementary with the target polynucleotide sequence. However, the probe need not necessarily be completely complementary to the target polynucleotide along the entire length of the target polynucleotide so long as the probe can bind the target polynucleotide with specificity and capture it from the sample.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by the skilled artisan. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. Determining appropriate hybridization conditions to identify and/or isolate sequences containing high levels of homology is well known in the art. For the purposes of specifying conditions of high stringency, preferred conditions are a salt concentration of about 200 mM and a temperature of about 45° C.

Data mining work is completed by bioinformatics, including scanning chips, signal acquisition, image processing, normalization, statistic treatment and data comparison as well as pathway analysis. As such, microarray can profile hundreds and thousands of polynucleotides simultaneously with high throughput performance. Microarray profiling analysis of mRNA expression has successfully provided valuable data for gene expression studies in basic research. And the technique has been further put into practice in the pharmaceutical industry and in clinical diagnosis. With increasing amounts of miRNA data becoming available, and with accumulating evidence of the importance of miRNA in gene regulation, microarray becomes a useful technique for high through-put miRNA studies.

The analysis of miRNA correlated with cancer can be carried out separately or simultaneously with multiple polynucleotide probes within one test sample. For example, several probes can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in miRNA levels over time. Increases or decreases in miRNA levels, as well as the absence of change in levels, can provide useful information about the disease status.

In some embodiments, a panel consisting of polynucleotide probes that selectively bind cancer-derived microvesicle miRNAs correlated with one or more cancers can be constructed to provide relevant information related to the diagnosis or prognosis of cancer and management of subjects with cancer. Such a panel can be constructed, for example, using 1, 2, 3, 4, 5, 6, 7, 8, 9, 10, 15, 20, 30, 40, 50, 75, 100, 150, 200, 250, 300, 400, 500, or 1,000 individual polynucleotide probes. In some cases, a panel comprises more than 1,000 individual polynucleotide probes. The analysis of a single probe or subsets of probes comprising a larger panel of probes could be carried out by one skilled in the art to optimize clinical sensitivity or specificity in various clinical settings. These include, but are not limited to ambulatory, urgent care, critical care, intensive care, monitoring unit, in-subject, out-subject, physician office, medical clinic, and health screening settings. Furthermore, one skilled in the art can use a single probe or a subset of additional probes comprising a larger panel of probes in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts.

In some embodiments, determining the amount of the one or more miRNAs comprises labeling the one or more miRNAs. The labeled miRNAs can then be captured with one or more polynucleotide probes that each selectively bind the one or more miRNAs.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into/onto a molecule, such as a polynucleotide. Various methods of labeling polypeptides are known in the art and can be used. Examples of labels for polynucleotides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for antibodies, metal binding domains, epitope tags, etc.). In some embodiments, labels are attached by spacer arms of various lengths to reduce potential steric hindrance.

The analysis of miRNA levels using polynucleotide probes can be carried out in a variety of physical formats as well. For example, the use of microtiter plates or automation can be used to facilitate the processing of large numbers of test samples. Alternatively, single sample formats could be developed to facilitate immediate treatment and diagnosis in a timely fashion.

In some embodiments, the plurality of polynucleotide probes are each bound to a substrate. In some embodiments, the substrate comprises a plurality of addresses. Each address can be associated with at least one of the polynucleotide probes of the array. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” miRNA can be referenced as a moiety in a mobile phase (typically fluid), to be detected by probes (“target probes”) which are bound to the substrate at the various regions.

Biopolymer arrays (e.g., polynucleotide microarrays) can be fabricated by depositing previously obtained biopolymers (such as from synthesis or natural sources) onto a substrate, or by in situ synthesis methods. Methods of depositing obtained biopolymers include, but are not limited to, loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. Further details of fabricating biopolymer arrays by depositing either previously obtained biopolymers or by the in situ method are disclosed in U.S. Pat. Nos. 6,242,266, 6,232,072, 6,180,351, and 6,171,797. In fabricating arrays by depositing previously obtained biopolymers or by in situ methods, typically each region on the substrate surface on which an array will be or has been formed (“array regions”) is completely exposed to one or more reagents. For example, in either method the array regions will often be exposed to one or more reagents to form a suitable layer on the surface that binds to both the substrate and biopolymer or biomonomer. In in situ fabrication the array regions will also typically be exposed to the oxidizing, deblocking, and optional capping reagents. Similarly, particularly in fabrication by depositing previously obtained biopolymers, it can be desirable to expose the array regions to a suitable blocking reagent to block locations on the surface at which there are no features from non-specifically binding to target.

Determining the amount of cancer-derived microvesicle miRNAs can alternatively, or in addition to microarray analysis, comprise using real-time polymerase chain reaction (PCR), for example such as is disclosed in detail in the present Examples. Real-time PCR (RT-PCR) can provide accurate and rapid data as to presence and amount of miRNAs present in a sample. FIG. 2 provides a flowchart of an exemplary protocol for isolating and measuring microvesicle-derived miRNAs by RT-PCR. Additional details of exemplary methodologies are set forth in the present Examples.

In some embodiments, the methods of the invention comprise providing a biological sample from a subject and isolating microvesicles comprising micro-RNAs (miRNAs) from the biological sample. The biological sample can be a bodily fluid such as described herein, e.g., plasma or serum. An amount of one or more of the miRNAs is then determined and compared to one or more miRNA control levels. The subject can then be diagnosed with having or being at risk of a head and neck cancer if there is a measurable difference in the amount of the one or more miRNAs from the microvesicles as compared to the one or more miRNA control levels. The levels of the one or more miRNAs can also be used to provide a prognosis or a theranosis, such as to classify the subject as a likely responder or non-responder to a treatment or to monitor the efficacy of a treatment over time. As such, in some embodiments, methods can include predicting response to a treatment in a subject, or predicting non-response of a treatment in a subject. The control levels can be the levels of the one or more miRNAs in a control sample that does not have or is not at risk of having a head and neck cancer, e.g., the control sample can be from a healthy subject. When monitoring one or more miRNA levels over time, a control can also be the level of the one or more miRNAs at a different time point. For example, a decrease in the level of one or more miRNA in a subject over time may indicate a response to a treatment.

The one or more miRNA that is assessed can include without limitation one or more of let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32 and miR-181c. In an embodiment, the miRNAs that are detected comprise one or more of miR-16, miR-181c, miR-25, miR-15b, miR-150, miR-148b, miR-92a, miR-222, miR-96, miR-125a-5p, miR-335 and miR-122. In another embodiment, the miRNAs that are detected comprise one or more of let7a, miR133b, miR122, miR20b, miR335, miR196a, miR125a-5p, miR142-5p, miR96, miR222, miR148b, miR92a, miR214, miR130a, miR29a, miR212, miR124, miR21, miR200c, miR100, miR155, miR181b, and miR210.

The presently-disclosed subject matter is inclusive of uses of reagents as described herein and reagents known to those of ordinary skill in the art to carry out the methods as disclosed herein and described in the claims. The presently-disclosed subject matter further includes kits that include reagents as described herein and reagents known to those of ordinary skill in the art to carry out the methods as disclosed herein and described in the claims.

The presently-disclosed subject matter further includes systems and kits, which are useful for practicing embodiments of the methods as described herein. In some embodiments a kit is provided, which is useful for determining a presence or an amount of one or more micro RNAs, which includes a probe for determining the presence or amount of each of one or more mircroRNAs in a sample. In some embodiment, the probe(s) are polynucleotides. In some embodiments a primer pair is used to determine the amount of the one or more microRNAs. In some embodiments, the probe(s) is provided on a substrate. In some embodiments the kit includes a probe for each of at least 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 miRNAs.

In some embodiments, kits of the presently-disclosed subject matter further include a reference standard sample to obtain a presence or amount of the one or more microRNAs for use as a control to which the sample (e.g., sample from the subject) can be compared. In some embodiments, the systems further include control data of a presence or level of the one or more microRNAs for use as a control to which the sample (e.g., sample from the subject) can be compared. In some embodiments, the systems further include reference data for one or more clinicopathologic features useful for characterizing a cancer-of-interest.

In some embodiments, the standard sample or the control data can be selected from: a standard sample or control data for head and neck cancer; a standard sample or control data for head and neck squamous cell carcinoma; a standard sample or control data for lung cancer; a standard sample or control data for lung adenocarcinoma; a standard sample or control data for lung squamous cell carcinoma; a standard sample or control data for non-cancer; a standard sample or control data for a responder; and a standard sample or control data for a nonresponder.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

In certain instances, microRNAs (miRNAs) disclosed herein are identified with reference to names assigned by the miRBase Registry (available at www.mirbase.org). The sequences and other information regarding the identified miRNAs as set forth in the miRBase Registry are expressly incorporated by reference as are equivalent and related miRNAs present in the miRBase Registry or other public databases. Also expressly incorporated herein by reference are all annotations present in the miRBase Registry associated with the miRNAs disclosed herein. Unless otherwise indicated or apparent, the references to the miRBase Registry are references to the most recent version of the database as of the filing date of this application (i.e., mirBase 19, released Aug. 1, 2012).

While the terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter. The following examples may include compilations of data that are representative of data gathered at various times during the course of development and experimentation related to the present invention

The presently disclosed subject matter discloses that miRNA can be found and isolated from microvesicles in biological fluids. The isolated miRNA can be used as a diagnostic tool for disorders such as cancer. The present Examples provide support for these applications.

### Example 1

### Materials and Methods

The following techniques are used to carry out the methods of the present invention.

Isolation of Circulating Vesicles

Microvesicles in a biological sample such as serum can be isolated using methods known in the art and/or disclosed herein, including centrifugation, PEG-precipitation or chromatographic isolation. See, e.g., isolation methods described in United States Patent Publication US 2010/0151480 A1, entitled “Exosome-associated MicroRNA as a Diagnostic Marker” and published on Jun. 17, 2000; and Taylor et al., “Chapter 15: Exosome Isolation for Proteomic Analyses and RNA Profiling,” in Richard J. Simpson and David W. Greening (eds.), Serum/Plasma Proteomics: Methods and Protocols, Methods in Molecular Biology, vol. 728, pp 235-246, © Springer Science+Business Media, LLC 2011, which publications are incorporated by reference herein in their entirety.

To isolate microvesicles by ultracentrifugation, the biological fluid (2 ml) is centrifuged at 12,000×g for 15 minutes. This supernatant is centrifuged at 100,000×g for 1 hour at 4° C. The pellet containing vesicles is resuspended in PBS and then re-centrifuged for 1 hour at 4° C. The vesicle pellet is extracted using a Trizol extraction procedures for RNA and protein analyses.

To isolate microvesicles by size exclusion chromatography, 2 ml aliquots of biological fluid are applied to a Sepharose 2B column (2.5×16 cm), eluted with PBS and 2 ml fractions collected, monitoring elution at 280 nm. The void volume fractions containing vesicles are pooled and centrifuged at 100,000×g for 1 h. The vesicle pellet is extracted using a Trizol extraction procedures for RNA and protein analyses. Chromotography systems such as the Bio-Rad Biologic chromatography system can be used (Bio-Rad, Hercules, Calif.).

To isolate vesicles by PEG precipitation, the biological fluid (2 ml) is transferred to a sterile tube and 0.5 ml of ExoQuick precipitation solution (System Biosciences (SBI), Mountain View, Calif.) is added and mixed. The mixture is incubated overnight (at least 12 hours) at 4° C. and then the mixture centrifuged at 12,000 rpm in a microfuge for 5 minutes. The supernatant is aspirated and the vesicle pellet is extracted using a Trizol extraction procedures for RNA and protein analyses.

Tumor-derived microvesicles are specifically isolated by a modified magnetic activated cell sorting (MACS) procedure, using an antibody to a microvesicle surface protein that is associated with cancers, such as anti-epithelial cell adhesion molecule (EpCAM). Other microvesicle markers are known in the art and can be used to capture microvesicles, such as tetraspanins such as CD9 and/or CD63. Serum samples (2.5 ml) from normal controls, patients with benign disease, and patients with cancer are incubated with antibodies to a microvesicle surface protein coupled to magnetic microbeads (50 μl). These are mixed and incubated for 2 hrs at 4° C. A LD microcolumn is placed in the magnetic field of a MACS Separator and the column is rinsed with 500 μl Tris-buffered saline (TBS). The magnetic immune complexes are applied onto the column and unbound (unlabeled) material that passes through is discarded. The column is washed four times with 500 μl of TBS. The specifically selected microvesicles are recovered by removing the column from the separator and placing it on a collection tube. TBS (1 ml) is added to the column and the magnetically labeled microvesicles are obtained by applying the plunger supplied with the column. The isolated microvesicles/microbeads are diluted in IgG elution buffer (Pierce Chemical Co, Rockford, Ill.) and the complex is centrifuged at 10,000 rpm to separate the microbeads from the microvesicles (supernatant). The supernatant is then centrifuged at 100,000 g for 1 hour at 4° C. The pelleted microvesicles are resuspended in 250 μl phosphate-buffered saline (PBS) and these tumor derived microvesicles are assayed for total protein. The quantity of protein is determined by the Bradford microassay method (Bio-Rad Laboratories, Hercules, Calif.), using bovine serum albumin (BSA) as a standard. The microvesicles can be isolated using this method after vesicles have been non-specifically isolated from the biological sample as described above.

Transmission Electron Microscopy

For transmission electron microscopy, the pelleted microvesicles are fixed in 2.5% (w/v) glutaraldehyde in PBS, dehydrated and embedded in Epon. Ultrathin sections (65 nm) are cut and stained with uranyl acetate and Reynold's lead citrate. The sections are examined in a Jeol 1210 transmission electron microscope.

Isolation and Profiling of miRNA

Total RNA is isolated from tumor cells and microvesicles using the mirVana miRNA isolation kit according to manufacturer's instructions (Ambion, Austin, Tex.). The RNA quality, yield, and size of miRNA fractions are analyzed using Agilent 2100 Bioanalyzer (Agilent Technologies, Foster City, Calif.). The isolated miRNAs are 3′-end labeled with Cy3 using the mirVana miRNA Array Labeling Kit (Ambion) and the Post Labeling Reactive Dye kit (Amersham Bioscience, Pittsburgh, Pa.). MicroRNA profiling is performed in duplicate by Ocean Ridge Biosciences (Jupiter, Fla.) using microarrays containing probes for 467 human mature miRNAs. This analysis uses custom-developed miRNA arrays covering the 467 miRNAs present in the mirBASE v9.0, consisting of 35-44-mer oligonucleotides, manufactured by Invitrogen and spotted in duplicate. After hybridization, the miRNA arrays are scanned using a GenePix 4000A array scanner (Axon Instruments, Union City, Calif.) and the raw data is normalized and analyzed using GeneSpring 7.0 Software (Silicon Genetics, Redwood City, Calif.). Normalization is performed by expressing each miRNA replicate relative to control miRNA (Ambion) added to each sample, allowing comparisons between arrays. Threshold and 95^{th} percentile of negative controls (TPT95) are calculated based on hybridization signal from negative control probes including: 38 mismatch and shuffled control probes and 87 non-conserved C. elegans probes. To define sensitivity, NCode synthetic miRNA is spiked at 1/500,000 mass ratio into labeling reactions and the signal intensity is detected. For specificity, perfect match probes for miR-93, miR-27a, and miR-152 and 2 mismatches for each are used. Typically, the 2 base pair mismatch probes demonstrate a signal below or at TPT95 on all arrays.

Alternately, total RNA is isolated from microvesicles using Trizol according to manufacturer's instructions (Invitrogen). The RNA quality and yield is accessed using a GeneQuant II (Pharmacia). The distribution of the small RNAs is analyzed using an Agilent 2100 Bioanalyzer (Agilent Technologies, Foster City, Calif.).

Microvesicle Protein Analysis by SDS-PAGE and Western Immunoblotting

Microvesicle protein isolation is performed using the Trizol isolation procedure above, as described by the manufacturer. The quantity of protein is determined by the Bradford microassay method (Bio-Rad Laboratories, Hercules, Calif.), using BSA as a standard. SDS-PAGE is performed by the method of Laemmli (1970) and the separated proteins are visualized by protein staining using Imperial Purple (Pierce Chemical). Western immunoblotting is performed to analysis the presence of specific proteins, e.g., microvesicle markers such as tetraspanin CD63 and EpCAM. Proteins from each microvesicle isolate (40 μg) are applied per lane of a 4-20% SDS-PAGE gel. The proteins are electrophoretically separated by SDS-PAGE and analyzed by western immunoblot, probing overnight at 4° C. with primary antibody. The bound immune complexes are visualized by enhanced chemiluminescence (ECL, Amersham Life Sciences, Arlington Heights, Ill.) and quantitated by densitometry (Un-Scan-it Software, Silk Scientific Corp., Orem, Utah).

General Statistical Considerations

Data is analyzed using the statistical software package, SAS 9.1 (SAS Institute, Cary, N.C.). The levels of circulating microvesicles for each group of subjects is defined as mean±standard deviation from at least two separate experiments performed in triplicate. Comparisons between groups is performed by one-way ANOVA, followed by the Tukey's multiple comparisons post-test comparing each population. Relative quantification of miRNA expression is calculated with the 2^{-ΔΔCt} method (Applied Biosystems User Bulletin No. 2) and data is analyzed as log 10 of relative quantity (RQ) of the target miRNA, normalized with respect to control miRNA added to each sample, allowing comparisons between arrays. The miRNA distributions and correlations along with confidence intervals are calculated for each subset. Statistical significance is set as p≦0.05.

### Example 2

### Microvesicle-miRNA Profiles in Squamous Cell Carcinoma and Adenocarcinoma

Predicting individual responses of cancer to treatment remain challenging, and diverging clinical courses of same cancer stage remain obscure. Better methods of defining cancer are needed. In this Example, blood-borne miRNA was reported to identify head and neck squamous cell carcinoma (HNSCC), lung SCC, lung adenoma and to predict outcome.

Heparinized blood samples were obtained from patients diagnosed with HNSCC (n=32) at the time of initial treatment and serial samples were also obtained during clinical follow-up, under an IRB approved protocol. Samples were also obtained from normal (i.e., non-cancer) patients, and patients with diagnosed lung SCC and lung adenocarcinoma. The blood samples were centrifuged at 400×g for 10 minutes to separate remove cells. The plasma was then centrifuged at 15,000×g for 20 minutes to remove cell debris. Circulating microvesicles were isolated by chromatography, following by precipitation by ExoQuick™ (System Biosciences, Mountain View, Calif.). Total RNA was extracted by a modified Trizol protocol and small RNA isolated using a small RNA isolation kit (SABiosciences, a Qiagen company, Frederick, Md.).

Eighty-eight specific miRNAs within the small RNA were quantitated using a cancer specific qRT-PCR array (SABiosciences) with an Agilent M3005P (Agilent Technologies, Santa Clara, Calif.). Single-stranded cDNA was synthesized from 5.5 ng of total RNA in 15 μl reaction volume by using the TaqMan MicroRNA Reverse Transcription Kit (Applied Biosystems, by life technologies, Carlsbad, Calif.). The reactions were incubated first at 16° C. for 30 min and then at 42° C. for 30 min. The reactions were inactivated by incubation at 85° C. for 5 min. Each cDNA generated was amplified by quantitative PCR by using sequence-specific primers from the TaqMan microRNA Assays Human Panel on an Agilent M3005P. The 20 μl PCR mix included 10 μl of 2× Universal PCR Master Mix, 2 μl of each 10× TaqMan MicroRNA Assay Mix and 1.5 μl of reverse transcription (RT) product. The reactions were incubated at 95° C. for 10 min, followed by 40 cycles of 95° C. for 15 s and 60° C. for 1 min. The threshold cycle (C_{T}) was defined as the fractional cycle number at which the fluorescence passes the fixed threshold (0.2). All signals with C_{T}≧37.9 were manually set to undetermined. The relative quantity (RQ) of the target miRNAs was estimated by the ΔC_{T} study by using as reference (exogenous control) for each preparation. Each sample was run in duplicate and each PCR experiment included two non-template control wells. For comparison, fold changes were defined by comparison to those obtained using normal human AB serum.

For miRNA levels, normalization is used for the accurate quantification and several approaches have been examined to normalize expression data. Relative quantification of miRNA expression was calculated with the 2^{-ΔΔCT} method (Applied Biosystems User Bulletin N°2) and data were presented as log 10 of relative quantity (RQ) of target miRNA, normalized with respect to miR-92. A second normalization approach showed the data as log 10 of relative quantity (RQ) of target miRNA, normalized to sn/snoRNA and relative to control sample. Finally, similar to microarray data, raw data C_{T} were normalized and analyzed using BRB ArrayTools version 3.3.2 (BRB-ArrayTools is developed by Dr. Richard Simon and BRB-ArrayTools Development Team and available from the National Cancer Institute at linus.nci.nih.gov/BRB-ArrayTools.html). After global median normalization, normalized data were presented as log 10 of relative quantity (RQ) of target miRNA relative to a control sample. Class Comparison and Significant analysis of microarrays (SAM) was performed to identify differentially expressed miRNAs. Visualization of results was performed with the different normalized data using average linkage and Euclidean distance as a measurement of similarity using GENESIS Software (Stern et al., Genesis: cluster analysis of microarray data, Bioinformatics, 18:207-208).

Eighty four miRs were analyzed including let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32 and miR-181c. Expression of 12 miRs was observed in HNSCC patient samples, including miR-16, miR-181c, miR-25, miR-15b, miR-150, miR-148b, miR-92a, miR-222, miR-96, miR-125a-5p, miR-335 and miR-122. The miRNA expression was compared to those expressed in lung SCC and lung adenocarcinoma. See FIGS. 4A-C. FIG. 4D includes data for controls.

Thirty head and neck cancer patients entered in the study. Fifteen were considered for analysis since at least one follow-up sample was collected post treatment. Patients with HNSCC exhibited miRNA profiles within circulating microvesicles that were distinct from normal controls and patients with squamous cell carcinoma (SCC) of the lung. Of the 84 miRNA analyzed and 12 detected, miR148b and miR222 appeared to be uniquely expressed in HNSCC, while miR16 was present in microvesicles on patients with both HNSCC and lung SCC. Patients with lung SCC appear to uniquely express miR199a and miR200c. In patients responding to initial therapy, the levels of most microvesicle-miRNAs were suppressed; however, in patients failing to respond, no decrease in the microvesicle-miRNAs was observed. See FIGS. 5A-B.

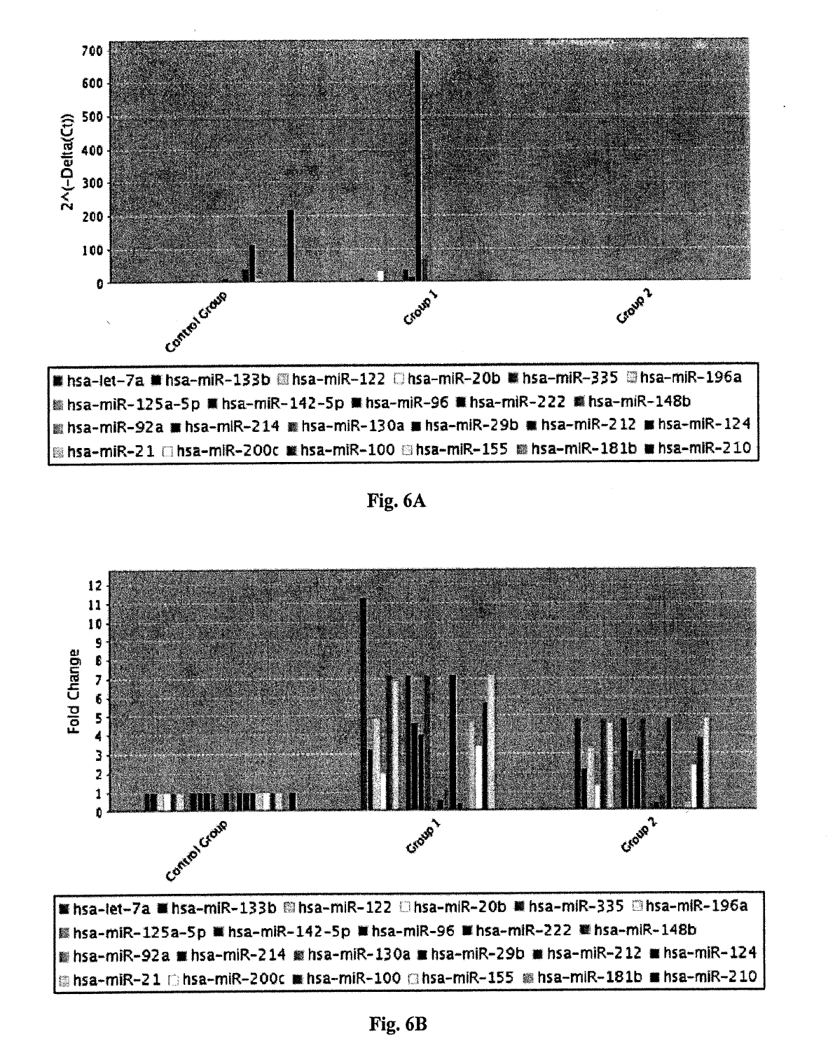

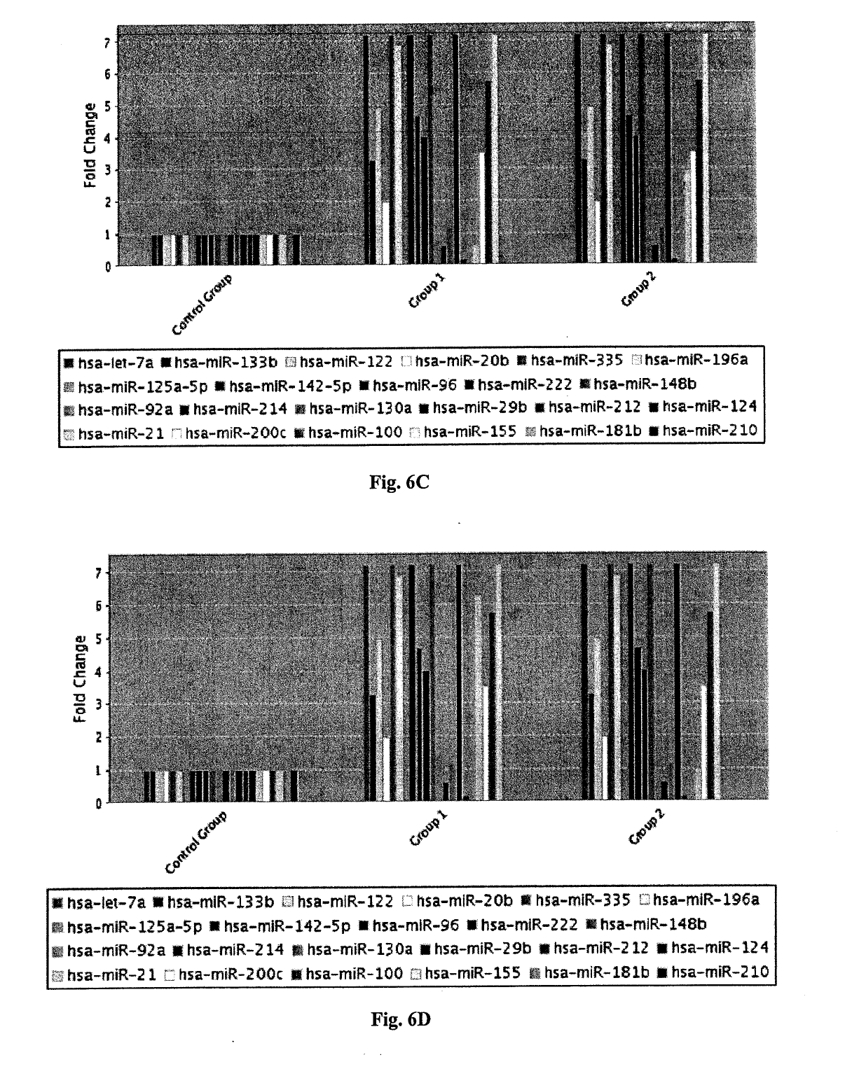

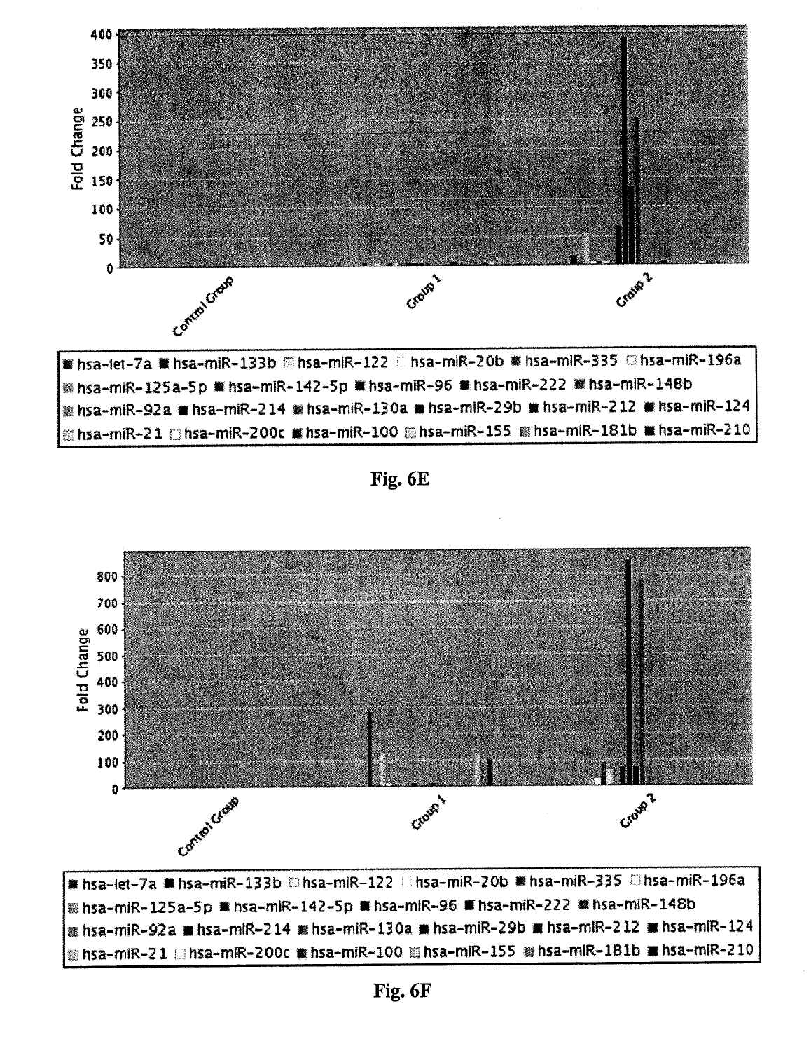

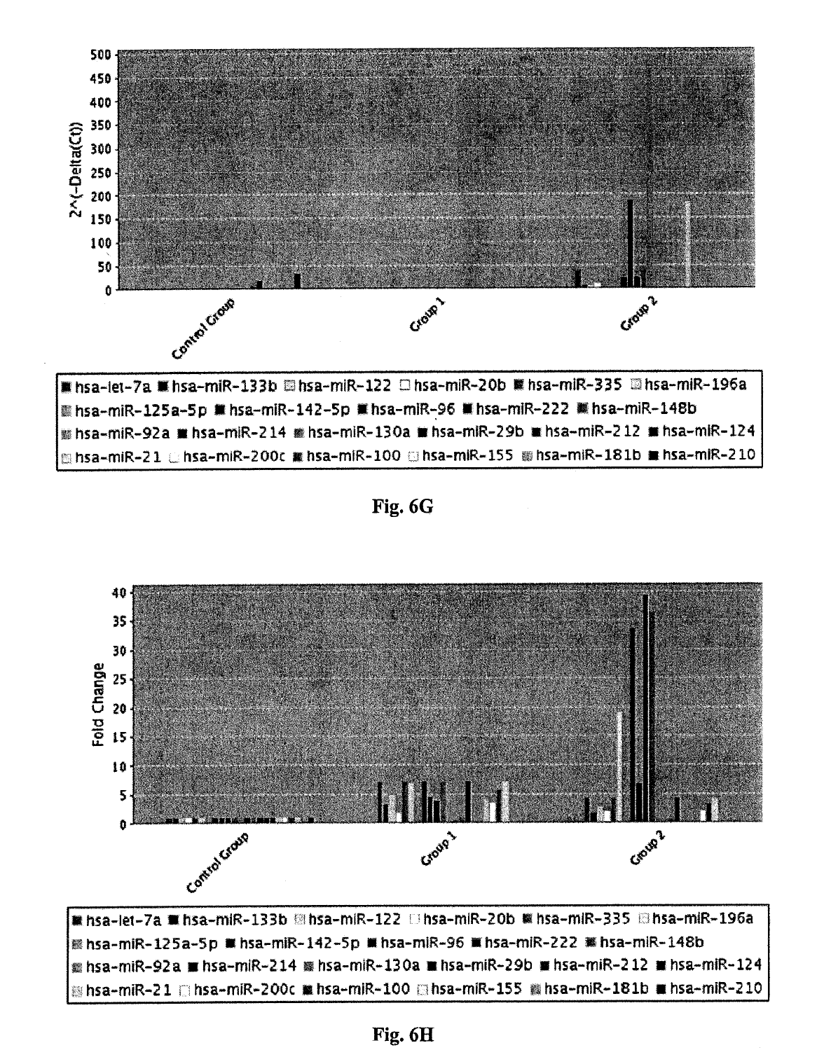

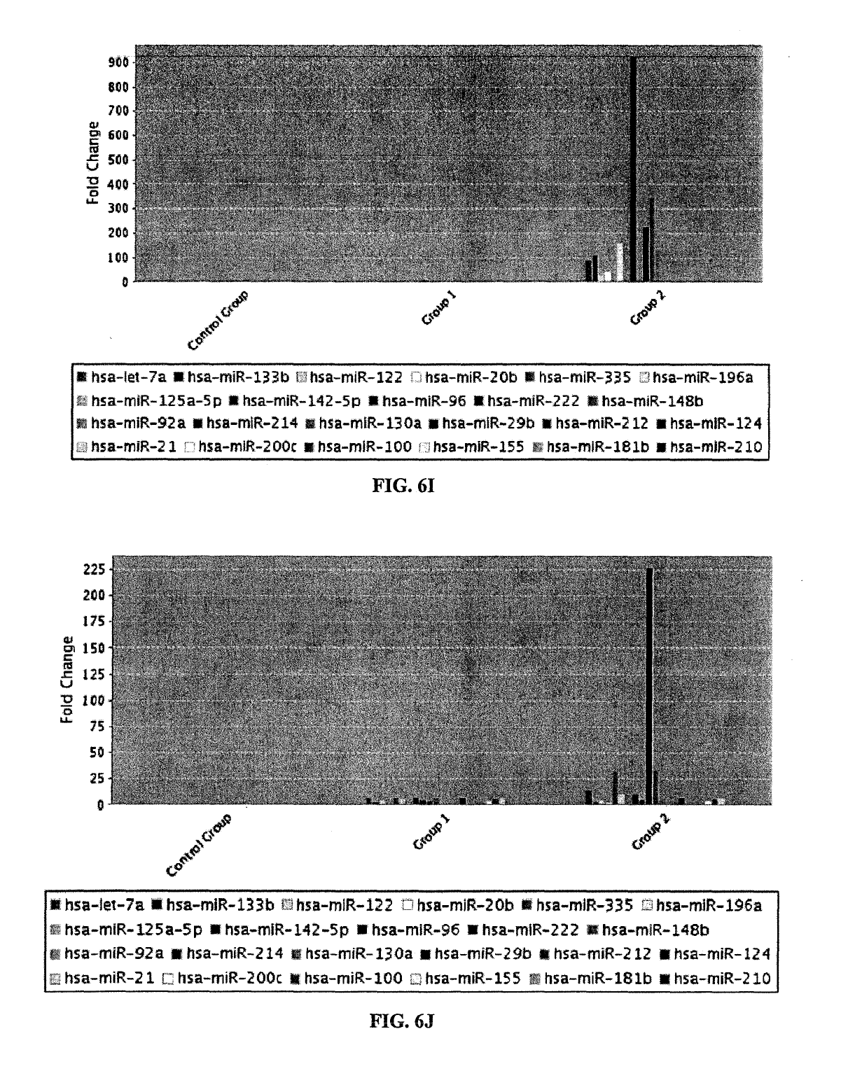

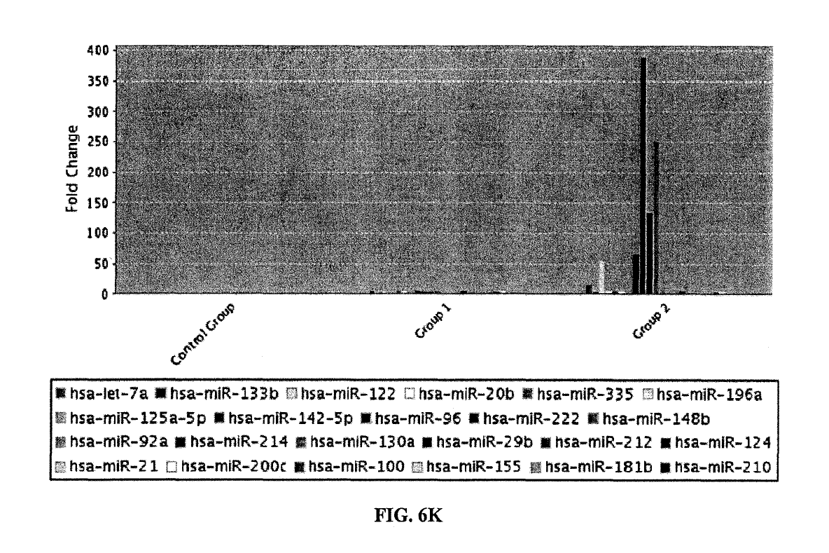

With reference to FIGS. 6A-K, miRNA profiles were obtained for the head and neck cancer patients before treatment and after treatment. Profiles for control (non-cancer) patients were also obtained. The pre-treatment sample was obtained on the day of, but prior to surgery (“Group 1”) and the post-treatment sample was obtained 3 months post surgery (“Group 2”). Treatment occurred following surgery, and included either chemotherapy or a combination of chemotherapy and radiation. Patients were identified as “responders” or “non-responders.” Patients considered responders have no evidence of disease at 18 months after initial surgery, while non-responders were diagnosed with recurrent disease within 18 months of initial surgery. The responders exhibited exhibited miRNA profiles within circulating microvesicles that were distinct from non responders, and both responders and non-responders exhibited miRNA profiles within circulating microvesicles that were distinct from normal controls.

miRNA profiles within blood-borne microvesicles have utility in the identification of cancers, including HNSCC, lung SCC and lung adenocarcinoma. In addition to their role in diagnosis, the microvesicle-miRNA profiles are useful for disease monitoring.

Throughout this document, various references are mentioned. All such references are incorporated herein by reference, including the references set forth in the following list:

### REFERENCES

1. Andre F, Schartz N E, Movassagh M, et al. Malignant effusions and immunogenic tumour-derived exosomes. Lancet 2002; 360: 295-305.
2. Bard M P, Hegmans J P, Hemmes A, et al. Proteomic analysis of exosomes isolated from human malignant pleural effusions. Am J Respir Cell Mol Biol 2004; 31:114-21.
3. Bartel D P. MicroRNAs: Genomics, biogenesis, mechanism, and function. Cell 2004; 116:281-97.
4. Berek J S, Schultes B C, Nicodemus C F. Biologic and immunologic therapies for ovarian cancer. J Clin Oncol 2003; 21(s10):168-74.
5. Calin G A, Croce C M. MicroRNA-cancer connection: the beginning of a new tale. Cancer Res 2006a; 66:7390-94.
6. Calin G A, Croce C M. MicroRNA signatures in human cancers. Nature Rev Cancer 2006b; 6:857-66.
7. Choi D S, Lee J M, Park G W, et al. Proteomic analysis of microvesicles derived from human colorectal cancer cells. J Proteome Res 2007; 6:4646-55.
8. De Cecco L, Marchionni L, Gariboldi M, Reid J F, Lagonigro M S, Caramuta S, et al. Gene expression profiling of advanced ovarian cancer: Characterization of a molecular signature involving fibroblast growth factor 2. Oncogene 2004; 23:8171-83.
9. Esquela-Kerscher A, Slack F J. Oncomirs-microRNAs with a role in cancer. Nature Rev Cancer 2006; 6:259-69.
10. Heijnen H F G, Schiel A E, Fijnheer R, Geuze H J, Sixma J J. Activation platelets release two types of membrane vesicles: Microvesicles by surface shedding and exosomes derived from exocytosis of multivesicular bodies and alpha granules. Blood 1999; 94:3791-9.
11. Iorio M V, Visone R, Di Leva G, Donati V, Petrocca F, Casalini P, et al. MicroRNA signatures in human ovarian cancer. Cancer Res 2007; 67:8699-707.
12. J. M. Escola J M, Kleijmeer M J, Stoorvogel W, Griffith J M, Yoshie O, Geuze H J. Selective enrichment of tetraspan proteins on the internal vesicles of multivesicular endosomes and on exosomes secreted by human B-lymphocytes. J Biol Chem 1998; 273: 20121-7.
13. Koga K, Matsumoto K, Akiyoshi T, Kubo M, et al. Purification, characterization and biological significance of tumor-derived exosomes, Anticancer Res 2005; 25: 3703-7.
14. Lu J, Getz G, Miska E A, Alvarez-Saavedra E, Lamb J, Peck D, et al. MicroRNA expression profiles classify human cancers. Nature 2005; 435: 834-8.
15. Mears R, Craven R A, Hanrahan S, et al. Proteomic analysis of melanoma-derived exosomes by two-dimensional polyacrylamide gel electrophoresis and mass spectrometry. Proteomics 2004; 4:4019-31.
16. Menon U, Jacobs U. Recent developments in ovarian cancer screening. Curr Opin Obstet Gynecol 2000; 12:39-42.
17. Miska E A. How microRNAs control cell division, differentiation, and death. Curr Opi. Genet Dev 2005; 5:563-8.
18. Olver C, Vidal M, Proteomic analysis of secreted exosomes. Subcell Biochem. 2007; 43:99-131.
19. Paul E. Blower P E, Chung J H, Verducci J S, Lin S, Park J K, Dai Z, Liu C G, Schmittgen T D, Reinhold W C, Croce C M, Weinstein J N, Sadee W. MicroRNAs modulate the chemosensitivity of tumor cells. Mol Cancer Therap 2008; 7: 1-9.
20. Raposo G, Tenza D, Mecheri S, Peronet R, Bonnerot C, Desaymard C. Accumulation of major histocompatibility complex class II molecules in mast cell secretory granules and their release upon degranulation. Mol Biol Cell 1997; 8:2631-45.
21. Ratajczak J, Miekus K, Kucia M, et al. Embryonic stem cell-derived microvesicles reprogram hematopoietic progenitors: Evidence for horizontal transfer of mRNA and protein delivery. Leukemia 2006; 20: 847-56.
22. Taylor D D, Doellgast G J. Quantitation of eroxidise-antibody binding to membrane fragments using column chromatography. Anal Biochem 1979; 98:53-9.
23. Taylor D D, Homesley H D, Doellgast G J. Binding of specific peroxidise-labeled antibody to placental-type alkaline phosphatase on tumor-derived membrane fragments. Cancer Res 1980:40:4964-69.
24. Taylor D D, Black P H. Shedding of plasma membrane fragments: Neoplastic and developmental importance. In: Developmental Biology, (M. Steinberg, ed.) vol. 3, 1986:33-57.
25. Taylor D D, Gercel-Taylor C. Tumour-derived exosomes as mediates of T-cell signaling defects. Brit Cancer 2005; 92:305-11.
26. Taylor, D. D., Bohler, H. C., Gercel-Taylor, C. Pregnancy-linked suppression of TcR signaling pathways by a circulating factor absent in recurrent spontaneous pregnancy loss. Molecular Immunology 2006, 43: 1872-80.
27. Valadi, H, Ekstrom K, Bossius A, Sjostrand M, Lee J J, Lotvall J O. Exosome-mediated transfer of mRNA and microRNA is a novel mechanism of genetic exchange. Nature Cell. Biol. 2007; 9:652-9.
28. Valenti R, Huber V, Filipazzi P, Pilla L, Sovena G, VIIIa A et al. Human tumor-released microvesicles promote the differentiation of myeloid cells with transforming growth factor-beta-mediated suppressive activity on T lymphocytes. Cancer Res 2006; 66: 9290-8.
29. Yang H, Kong W, He L, Zhao J J, O'Donnell J D, Wang J, Wenham W M, Coppola D, Kruk P A, Nicosia S V, Cheng J Q. MicroRNA expression profiling in human ovarian cancer: miR-214 induces cell survival and cisplatin resistance by targeting PTEN. Cancer Res 2008; 68: 425-33.
30. Zhang L, Huang J, Yang N, et al. microRNAs exhibit high frequency genomic alterations in human cancer. Proc Natl Acad Sci USA 2006; 103:9136-41.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for characterizing a lung cancer or a head and neck cancer in a subject, comprising:
a) isolating microvesicles from a biological sample of the subject;
b) determining a presence or an amount of one or more microRNAs from the isolated microvesicles; and
c) comparing the presence or the amount of the one or more microRNAs to a reference, wherein the lung cancer or the head and neck cancer is characterized based on a measurable difference in the presence or the amount of the one or more microRNAs from the isolated microvesicles as compared to the reference.

2-3. (canceled)

4. A method for assessing a presence or an amount of one or more microRNAs of a lung cancer miRNA signature or a head and neck cancer miRNA signature, comprising:
a) isolating microvesicles from a biological sample; and
b) determining the presence or the amount of the one or more microRNAs in said microvesicles, wherein the microvesicles are shed from lung cancer or head and neck cancer cells.

5. (canceled)

6. The method of claim 4, wherein the biological sample comprises a cell culture sample.

7. (canceled)

8. The method of claim 1, and further comprising determining an expression profile of two or more microRNAs, and comparing the expression profile with a profile from a selected reference sample to determine the presence or the amount of two or more microRNAs in said microvesicles.

9. The method of claim 1, wherein the reference comprises: a level of the one or more microRNAs in one or more samples from one or more individuals without the cancer; a level of the one or more microRNAs in a sample from the subject taken over a time course; a standard sample; and/or control data.

10-14. (canceled)

15. The method of claim 1, wherein isolating the microvesicles comprises: using size exclusion chromatography; PEG-precipitation of the microvesicles; centrifuging a chromatography fraction comprising the microvesicles; and/or affinity selection using a binding agent to a microvesicle surface antigen.

16. (canceled)

17. The method of claim 15, wherein isolating the microvesicles comprises centrifuging a chromatography fraction comprising the microvesicles and the chromatography fraction is a void volume fraction.

18. (canceled)

19. The method of claim 15, wherein isolating the microvesicles comprises affinity selection using a binding agent to a microvesicle surface antigen and the microvesicle surface antigen is a known cancer marker.

20. (canceled)

21. The method of claim 19, wherein the binding agent is an anti-epithelial cell adhesion molecule (anti-EpCAM) antibody, an anti-CD9 antibody, or an anti-CD63 antibody.

22-25. (canceled)

26. The method of claim 1, wherein the cancer is selected from a squamous cell carcinoma, an adenocarcinoma, a head and neck cancer, a head and neck squamous cell carcinoma, a lung cancer, a non-small cell squamous cell carcinoma a lung squamous cell carcinoma, and a lung adenocarcinoma.

27-34. (canceled)

35. The method of claim 1, wherein the one or more microRNAs comprises one or more of let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32 and miR-181c.

36. (canceled)

37. The method of claim 35, wherein the one or more microRNAs comprises one or more of miR-16, miR-181c, miR-25, miR-15b, miR-150, miR-148b, miR-92a, miR-222, miR-96, miR-125a-5p, miR-335 and miR-122, and overexpression of the one or more microRNAs as compared to the reference indicates the presence of a head and neck cancer in the subject.

38-39. (canceled)

40. The method of claim 35, wherein the one or more microRNAs comprises one or more of let7a, miR133b, miR122, miR20b, miR335, miR196a, miR125a-5p, miR96, miR92a, let 7b, miR21, miR150, miR15b, miR25, miR181c, miR199a-3p, miR200c, and miR16, and overexpression of the one or more microRNAs as compared to the reference indicates the presence of a lung cancer in the subject.

41. (canceled)

42. The method of claim 35, wherein the one or more microRNAs comprises one or more of let7a, miR133b, miR122, miR20b, miR335, miR196a, miR125a-5p, miR96, miR92a, let 7b, miR21, miR150, miR15b, miR25, and miR181c, and overexpression of the one or more microRNAs as compared to the reference indicates the presence of a lung adenocarcinoma in the subject.

43. (canceled)

44. The method of claim 35, wherein the one or more microRNAs comprises one or more of let7a, miR122, miR335, miR196a, miR125a-5p, miR96, miR92a, let 7b, miR21, miR150, miR15b, miR25, miR181c, miR199a-3p, miR200c, and miR16, and overexpression of the one or more microRNAs as compared to the reference indicates the presence of a lung squamous cell carcinoma in the subject.

45. (canceled)

46. The method of claim 1, further comprising selecting a treatment or modifying a treatment for the cancer based on the amount of the one or more microRNAs determined.

47. The method of claim 1, wherein response to a treatment in the subject is predicted.

48. The method of claim 47, wherein nonresponse to the treatment in the subject is predicted.

49-52. (canceled)

53. A kit comprising one or more primer pair for determining the amount of the one or more micro RNAs selected from: let-7a, miR-133b, miR-122, miR-20b, miR-335, miR-196a, miR-125a-5p, miR-142-5p, miR-96, miR-222, miR-148b, miR-92a, miR-184, miR-214, miR-15a, miR-18b, miR-378, let-7b, miR-205, miR-181a, miR-130a, miR-199a-3p, miR-140-5p, miR-20a, miR-146b-5p, miR-132, miR-193b, miR-183, miR-34c-5p, miR-30c, miR-148a, miR-134, let-7g, miR-138, miR-373, let-7c, let-7e, miR-218, miR-29b, miR-146a, miR-212, miR-135b, miR-206, miR-124, miR-21, miR-181d, miR-301a, miR-200c, miR-100, miR-10b, miR-155, miR-1, miR-363, miR-150, let-7i, miR-27b, miR-7, miR-127-5p, miR-29a, miR-191, let-7d, miR-9, let-7f, miR-10a, miR-181b, miR-15b, miR-16, miR-210, miR-17, miR-98, miR-34a, miR-25, miR-144, miR-128, miR-143, miR-215, miR-19a, miR-193a-5p, miR-18a, miR-125b, miR-126, miR-27a, miR-372, miR-149, miR-23b, miR-203, miR-32 and miR-181c.

54. The kit of claim 53, and further comprising a reference standard sample and/or control data for head and neck cancer, head and neck squamous cell carcinoma, lung cancer, lung adenocarcinoma, lung squamous cell carcinoma, and/or non-cancer.

55-64. (canceled)
